(19) United States
(12) Patent Application Publication
(10) Pub. No.: US 2010/0324355 A1
(43) Pub. Date: Dec. 23, 2010

(54) DEVICE AND METHOD FOR IMPROVING HEARING

(75) Inventors: Hartmut H.R. Spitaels, Lubbeek (BE); Koenraad F.C. Van Schuylenbergh, Lille (BE); Stephanus A.E. Peeters, Aartselaar (BE)

Correspondence Address: KNOBBE MARTENS OLSON & BEAR LLP, 2040 MAIN STREET, FOURTEENTH FLOOR, IRVINE, CA, 92614, US

(73) Assignee: 3WIN N.V., Niel (BE)

(21) Appl. No.: 12/521,545

(22) PCT Filed: Dec. 21, 2007
(86) PCT No.: PCT/EP2007/064462

§ 371 Date: Jun. 26, 2009

(30) Foreign Application Priority Data
Dec. 26, 2006 (EP) PCT EP2006 012532

(51) Int. Cl. H04R 25/02; A61N 1/36

(52) U.S. Cl. 600/25; 607/57

(57) ABSTRACT

An implantable device for improving hearing is provided. The device includes a vibration generator including an output region configured to apply vibrational stimulation to an inner ear fluid, a proximal electrode configured to physically attach to a wall enclosing an inner ear at a location proximal to the output region of the vibration generator, and a separate distal electrode configured to make electrical contact with an auditory nerve.

### FIELD OF THE INVENTION

The invention is in the field of implantable hearing devices, a kit, and methods for the implantation of said hearing devices.

### BACKGROUND TO THE INVENTION

Sounds are perceived in humans by means of a mechanical-neural system distributed over the external ear canal, the middle ear cavity and the cochlea. Sound waves propagate through the external ear canal to reach and vibrate the tympanic membrane. The middle ear ossicles—malleus, incus and stapes—transfer the tympanic membrane vibrations to the footplate of the oval window that seals off the cochlea. Footplate vibrations set up waves of fluid motion within the fluid that is contained in the cochlea. The fluid motions in turn activate hair cells inside the cochlea. The hair cells produce in response electrical nerve impulses that are routed through the spiral ganglion and the auditory nerve to the brain, where they are perceived as sound. The electro-mechanics of the cochlear membranes and hair cells vary gradually along the length of the cochlea, which creates a natural spectral distribution of sensitivity along the cochlea: high-pitch sounds activate the hair cells near the oval window, whereas the lower pitches activate the hair cells further down the cochlea.

Modification and/or amplification of the energy reaching the sensory cells of the inner ear are the basis for treatment of conductive and sensorineural hearing losses. First attempts to improve hearing by making a hole in the wall of the inner ear at the level of the lateral semicircular canal were undertaken in 1914 in a procedure called fenestration. In fenestration, a trough-shaped window is made in the bony wall of the inner ear and is covered with transposed tympanic membrane. This connects the fluid spaces of the human inner ear directly to the outside world bypassing the dysfunctional middle ear. This procedure enables the sound energy to reach directly the membranous part of the inner ear and can result in an improvement of hearing by up to 30 dB.

Currently, when opening of the inner ear space is necessary, other safer and more effective surgical techniques have been developed. In patients with otosclerosis (immobility of the ossicular chain due to fixation of the stapes footplate), a small-hole fenestration in the stapes footplate is made, and a Teflon piston is transposed between the incus and the opening in the footplate after removal of the stapes superstructure. This procedure, albeit quite difficult technically, normalises the functional status of the conductive part of the middle ear and, in most cases, restores hearing to normal or quasi-normal.

The main drawback of the latter technique is that the fenestration of the inner ear remains open, which incurs the risk for inner ear infections. This may lead to meningitis or total hearing loss. A solution is to cover the fenestration with a piece of tissue, however, this has in the long term a tendency to re-ossify, which leads to diminishing results.

Hearing improvement can also be achieved by amplification of the energy reaching the sensory cells of the inner ear, using a variety of hearing aids. All these devices try to compensate for the diminished hearing acuity by amplification of the energy reaching the inner ear. They either amplify air sound waves, vibrate the ossicular chain, or vibrate the bones of the skull. However, application of any one of these devices has a number of important drawbacks including lack of aesthetic appeal, poor performance of conventional hearing aids due to feedback and distortion, limited indications and variable results in implantable hearing aids.

There have also been a few devices described in the literature, which employ a direct energy transfer to or from the inner ear. The advantage of these systems is that relatively little energy is required to achieve substantial amplifications and that the transducers can be very small. Some of these direct energy transfer devices are described below.

The Round Window Electromagnetic device (RWEM) realises coupling to the cochlear fluids through an intact round window membrane, which serves as the natural flexible interface between the middle and the inner ear. The RWEM uses a magnet, surgically placed onto the round window and an electromagnetic coil to induce vibration. This vibration is transmitted through an intact round window membrane to the cochlea's fluids. The RWEM device, however, would compromise the normal compliance of the round window membrane, which could induce a hearing loss.

Leysieffer describes in DE 39 40 632 an implantable hearing aid with either separate electromechanical stimulation or separate electrical stimulation.

Money (U.S. Pat. No. 5,782,744) proposed an implantable microphone encapsulated in a waterproof casing and placed at the round window in contact with the cochlear fluid, immersed in the cochlear fluid or placed in the middle ear and coupled to the inner ear fluid by a conduction tube. Such a microphone transmits the pressure variations induced in the inner ear by acoustic stimulation.

A cochlear implant bypasses the mechanical signal chain altogether, and provides direct electrical stimulation of the auditory neural system using an elongated electrode inserted in and following either the scala tympani or the scala vestibuli.

Hybrid electrical-mechanical systems have been described recently that complement the electrical stimulation of a cochlear implant with mechanical means to induce vibrations in the inner ear fluid. Electrical stimulation complementary to mechanical stimulation can be a significant advantage to certain otological pathologies. In case of locally damaged inner ear structures, mechanical stimulation can be ineffective at related frequencies. For example in patients with presbyacousis where the sensory cells (hair cells) for sensing the high frequencies are damaged and no longer function, the related neural structures are functional and can be electrically stimulated to transfer high-frequency acoustical signals. There are also many pathologies other than presbyacousis pathologies with high-frequency hearing loss. In general, electrical stimulation is necessary whenever “dead frequency regions” are present that cause sound distortion when only stimulated acoustically/mechanically.

Leysieffer (U.S. Pat. No. 6,697,674) describes the combination of a cochlear electrode with an implanted mechanical transducer that vibrates parts of the middle ear. The middle ear vibrations find their natural way to the inner ear via the stapes footplate in the oval window. Harrison (U.S. Pat. No. 6,754,537) describes a hybrid system for patients with severe high-frequency hearing loss but normal or near normal hearing for low frequencies. He combines a cochlear electrode that electrically stimulates the cochlea with the high-frequency audio content, and relies on the patient's natural hearing to pick up the low-frequency audio content. This low-frequency content is then provided mechanically by either a conventional external hearing aid, or a middle-ear mechanical transducer. Leysieffer describes in U.S. Pat. No. 6,565,503 an electrical cochlear electrode modified with miniature mechanical transducers distributed over the electrode's length to generate mechanical vibrations in the inner ear fluid.

A drawback of known hybrid electrical-mechanical devices for hearing aids is that their implantation is a highly invasive procedure causing irreparable damage to the residual hearing the patient may still have. This is because they are configured either as a conventional cochlear electrode in combination with a mechanical device, or as a cochlear electrode modified with intra-cochlear electromechanical converters that generate mechanical vibrations in the inner ear fluid. Both types have an elongated electrode that is inserted in the scala vestibuli or scala tympani. They penetrate deep into the cochlea through a hole in the bony cochlea wall, thereby risking damaging the fine features inside and destroying whatever residual hearing the patient may still have. Shortening and thinning the electrodes to preserve hearing is an area of intensive research. It is technically challenging and experiments have yet to show conclusive and consistent improvements, although full coverage for speech has been demonstrated on some patients with a 16-17 mm outer-wall electrode. More important, implanting short electrodes actually jeopardizes the patient's prospects for later upgrades to longer electrodes, e.g. in cases of progressive hearing loss. This is caused by tissue growth around the electrodes that tears during electrode removal and ruptures the fragile basilar membrane with it.

The present invention aims at overcoming the problems associated with conventional hearing implants, by providing an effective method and device which retains residual hearing.

It also aims to allow the surgeon to implant an electrical and mechanical stimulatory hearing aid in a single procedure, in those cases where he does not have the foreknowledge of which stimulation would be the most effective.

### FIGURE LEGENDS

FIG. 1: A functional diagram of the ear, showing a configuration of the present invention whereby the proximal electrode and vibration generator are implanted in a hole created near to oval window for accessing the scala vestibule.

FIG. 2: A functional diagram of the ear, showing a configuration of the present invention whereby the proximal electrode and vibration generator are implanted in the oval window.

FIG. 3: A functional diagram of the ear, showing the prior art arrangement of an electrode inserted in the scala vestibuli or scala tympani of the cochlea.

FIG. 4: A cross-section through an in situ vibration generator of the present invention.

FIGS. 5 to 18: A cross-section view depicting an in situ vibration generator and proximal electrode.

FIG. 19: A three dimensional view of a cochlea disposed with components of the present device.

FIG. 20: A schematic view of a configuration of a regulating unit.

FIGS. 21, 24 to 30: Cross-section views depicting an in situ vibration generator and proximal electrode, where the vibration generator comprises a first and second sub-frame connected by an vibration-energy conducting element.

FIG. 22: A functional diagram of the ear, showing a configuration of the present invention whereby the proximal electrode implanted in a hole created near to oval window for accessing the scala vestibule, and vibration generator comprises a first and second sub-frame connected by an vibration-energy conducting element, the first sub-frame housing the electromechanical actuator implanted in the mastoid.

FIG. 23: A functional diagram of the ear, showing a configuration of the present invention whereby the proximal electrode implanted in a hole created near to oval window for accessing the scala vestibule, and vibration generator comprises a first and second sub-frame connected by an vibration-energy conducting element, the first sub-frame housing the electromechanical actuator incorporated in the control unit.

FIG. 31: Exploded view of a revolute joint present in a vibrational energy conducting element that is a hinged link.

### SUMMARY OF SOME EMBODIMENTS OF THE INVENTION

One embodiment of the invention is an implantable device for improving hearing in a subject comprising:
a vibration generator (5) comprising an output region (19) configured to apply vibrational stimulation to the inner ear (2) fluid,
a proximal electrode (1) configured for physical attachment to a wall enclosing the inner ear (2) at a location proximal to the output region, and
a separate distal electrode (3) configured to make electrical contact with the auditory nerve (4).

Another embodiment of the invention is an implantable device as described above, wherein the vibration generator comprises:
an electromechanical actuator (20),
a vibrating surface (25) co-operatively connected to the electromechanical actuator (20), which provides vibrational energy, and
a frame (22) configured to position the vibrating surface (25) to direct vibrational energy therefrom to the output region (19).

Another embodiment of the invention is an implantable device as described above, wherein the frame (22) is configured for physical attachment to a wall enclosing the middle ear (6).

Another embodiment of the invention is an implantable device as described above, wherein the frame (22) is configured for physical attachment to
a wall enclosing the middle ear (6),
a wall enclosing the inner ear (2),
a walled interface between the middle (6) and inner ear (2),
a walled interface between the inner ear (2) and mastoid region, or
a wall of a cavity created in the mastoid region.

Another embodiment of the invention is an implantable device as described above, wherein the vibrating surface (25) is a flat surface co-operatively connected to the electromechanical actuator (20).

Another embodiment of the invention is an implantable device as described above, wherein the vibrating surface (25) is extended by an elongated member co-operatively connected to the electromechanical actuator (20).

Another embodiment of the invention is an implantable device as described above, wherein the frame (22) comprises a first sub-frame (22a) that supports the electromechanical actuator (20) and a second sub-frame (22b) provided with the output region (19) wherein the vibration energy from the electromechanical actuator (20) is directed to the output region (19) via a vibrational-energy conducting element (80).

Another embodiment of the invention is an implantable device as described above, wherein the conducting element (80) is a tube (84) adapted to contain a non-compressible liquid or gel (81).

Another embodiment of the invention is an implantable device as described above, wherein the conducting element (80) is a cable link, comprising a flexible cable (83) housed in a sleeve (82), which cable (83) is configured to move within the sleeve (82), while maintaining a coaxial relation therewith.

Another embodiment of the invention is an implantable device as described above, wherein the conducting element (80) is a non-flexible, elongated rod (85).

Another embodiment of the invention is an implantable device as described above, wherein the conducting element (80) is an adjustable telescopic slip link (89).

Another embodiment of the invention is an implantable device as described above, wherein the conducting element (80) is an adjustable hinged link (91).

Another embodiment of the invention is an implantable device as described above, wherein the second sub-frame (22b) forms a passage (72) having a receiving end (70) to receive vibrational energy from the conducting element (80), and a transmitting end (71) where vibrational energy is directed towards the inner ear fluid.

Another embodiment of the invention is an implantable device as described above, wherein the second sub-frame (22b) is disposed with the vibrating surface (25) in the passage (72), optionally in a region towards or at the transmitting end (71).

Another embodiment of the invention is an implantable device as described above, wherein the vibrating surface (25) is a flexible or flexibly suspended membrane (73) in sealing connection with the transmitting end (71) of the passage (72), and in hydraulic connection with the electromechanical actuator (20).

Another embodiment of the invention is an implantable device as described above, wherein the vibrating surface (25) is a flexibly suspended plate in mechanical connection with the electromechanical actuator (20)

Another embodiment of the invention is an implantable device as described above, wherein the vibrating surface (25) is formed from a sliding piston (75) in hydraulic or mechanical connection with the electromechanical actuator (20).

Another embodiment of the invention is an implantable device as described above, wherein the vibrating surface (25) comprises:
a flexibly suspended rigid membrane (105) in sealing connection with the transmitting end (71) of the passage (72), and in hydraulic connection with the electromechanical actuator (20), and
a pin (101) attached to said membrane (105).

Another embodiment of the invention is an implantable device as described above, wherein the first sub-frame (22a) is configured for physical attachment to:
a wall enclosing the middle ear (6) or
a wall of a cavity created in the mastoid region.

Another embodiment of the invention is an implantable device as described above, wherein the first sub-frame (22a) is incorporated within the housing of a regulating unit (7).

Another embodiment of the invention is an implantable device as described above, wherein the second sub-frame (22b) is configured for attachment at
a wall enclosing the inner ear (2),
a walled interface between the middle (6) and inner ear (2), or
a walled interface between the inner ear (2) and mastoid region.

Another embodiment of the invention is an implantable device as described above, wherein the electromechanical actuator (20) is an electromagnetic, piezoelectric, electrostatic or magnetostrictive actuator.

Another embodiment of the invention is an implantable device as described above, wherein at least part of the frame (22) or at least part of the vibrating surface (25) acts as the proximal electrode (1).

Another embodiment of the invention is an implantable device as described above, wherein the proximal electrode (1) and/or the distal electrode (1) is pin-shaped and is configured to diverge from a longitudinal centreline of a cochlea (4) lumen.

Another embodiment of the invention is an implantable device as described above, wherein the proximal electrode (1), the output region (19) and/or distal electrode (3) is configured to sit flush or recessed with the inside wall of the inner ear (2).

Another embodiment of the invention is an implantable device as described above, wherein the proximal electrode (1), the output region (19) and/or distal electrode (3) is configured to sit flush or recessed with the inside wall of the cochlea (4) lumen.

Another embodiment of the invention is an implantable device as described above, further comprising a regulating unit (7) configured to provide electrical signals to said electrodes and/or vibration generator, which signals represent sound information.

Another embodiment of the invention is an implantable device as described above, wherein the regulating unit (7) is configured to provide full audio frequency spectrum to the vibration generator (5).

Another embodiment of the invention is an implantable device as described above, wherein the regulating unit (7) is configured to enhance or suppress one or more bands of audio frequency provided to the vibration generator (5).

Another embodiment of the invention is an implantable device as described above, wherein the regulating unit (7) is configured to translate sound information into electrical signals for triggering nerves to fire neural signals, which electrical signals are provided to the electrodes (1, 3).

Another embodiment of the invention is an implantable device as described above, wherein the regulating unit (7) is configured to translate full audio frequency spectrum into said signals.

Another embodiment of the invention is an implantable device as described above, wherein the regulating unit (7) is configured to enhance or suppress one or more bands of audio frequency and translate it into said signals.

Another embodiment of the invention is an implantable device as described above, wherein the regulating unit (7) is configured to split sound information into higher frequency signals and lower frequency signals, whereby the higher frequency signals are provided to the electrodes (1, 3) and the lower frequency signals are translated and provided to the vibration generator (5).

Another embodiment of the invention is an implantable device as described above, wherein the regulating unit (7) is configured to receive sound information from an internal microphone, an external microphone or a telecoil.

Another embodiment of the invention is an implantable device as described above, wherein the regulating unit (7) is configured to use measurements from a measurement electrode for closed-loop control of electrical and/or vibrational stimulation.

Another embodiment of the invention is an implantable device as described above, wherein the wherein the regulating unit (7) is configured to use readings from the electromechanical actuator (20) operating as a microphone for closed-loop control of electrical and/or vibrational stimulation.

Another embodiment of the invention is an implantable device as described above, wherein the wherein the regulating unit (7) is configured to generate also a static pressure using the vibration generator (5).

Another embodiment of the invention is an implantable device as described above, wherein the electromechanical actuator (20) is configured to act as a pressure sensor.

Another embodiment of the invention is an implantable device as described above, wherein the wherein the regulating unit (7) is configured to control an inner ear (2) pressure using the vibration generator (5).

Another embodiment of the invention is an implantable device as described above, wherein the regulating unit (7) comprises a receiving means configured to receive sound information across a wireless link.

Another embodiment of the invention is an implantable device as described above, wherein the regulating unit (7) comprises a transmitting and/or receiving means, configured to exchange data with an external device across a wireless link.

Another embodiment of the invention is an implantable device as described above, wherein the regulating unit (7) comprises memory storage configured to store patient-specific data.

Another embodiment of the invention is an implantable device as described above, wherein the distal electrode is disposed within the regulating unit (7).

Another embodiment of the invention is a method for improving hearing in a subject comprising the steps of:
implanting a vibration generator (5), comprising an output region (19) such that said output region is located in a wall enclosing the inner ear, and applies vibrational stimulation to the inner ear fluid,
implanting in a wall enclosing the inner ear (2), a proximal electrode (1), which electrode is proximal to the output region (19) of vibration generator (5),
implanting a distal electrode (3) such that it makes electrical contact with the cochlea (4).

Another embodiment of the invention is a method as described above, wherein the vibration generator further comprises:
an electromechanical actuator (20),
a vibrating surface (25) co-operatively connected to the electromechanical actuator (20), which provides vibrational energy, and
a frame (22) configured to position the vibrating surface (25) so as to direct vibrational energy therefrom to the output region (19).

Another embodiment of the invention is a method as described above, wherein the frame (22) of the vibration generator (5) is attached to the locations defined above.

Another embodiment of the invention is a method as described above, wherein the frame (22) of the vibration generator (5) is attached to a wall enclosing the middle ear (6).

Another embodiment of the invention is a method as described above, wherein the frame (22) of the vibration generator (5) is attached at the interface (28) between the middle (6) and inner ear (2).

Another embodiment of the invention is a method as described above, wherein the frame (22) is embedded in a cavity machined in a bony wall enclosing the middle ear (6), which wall is not an interface (28) between the middle (6) and inner ear (2).

Another embodiment of the invention is a method as described above, wherein said bony wall enclosing the middle ear (6) is the mastoid or temporal bone.

Another embodiment of the invention is a method as described above, wherein the frame (22) of the vibration generator (5) is attached so as to position the output region (19) in a hole drilled all the way through, or drilled partially through the interface (28) between the middle (6) and inner ear (2).

Another embodiment of the invention is a method as described above, wherein the frame (22) of the vibration generator (5) is attached so as to position the output region (19) in a hole drilled all the way through, or drilled partially through a wall enclosing the inner ear (2), preferably interface (28) between the middle (6) and inner ear (2), or preferably the interface between the inner ear (2) and the mastoid region.

Another embodiment of the invention is a method as described above, wherein said hole is in a bony part.

Another embodiment of the invention is a method as described above, wherein the frame comprises a first sub-frame (22a) that supports the electromechanical actuator (20) and a second sub-frame (22b) provided with the output region (19) as defined above.

Another embodiment of the invention is a method as described above, wherein the first sub-frame (22a) is attached to the locations defined above.

Another embodiment of the invention is a method as described above, wherein the first sub-frame (22a) is incorporated within the housing of a regulating unit (7).

Another embodiment of the invention is a method as described above, wherein the second sub-frame (22b) attached the locations defined above.

Another embodiment of the invention is a method as described above, wherein the proximal electrode (1) is implanted at the interface between the middle (6) and inner ear (2).

Another embodiment of the invention is a method as described above, wherein the proximal electrode (1) is implanted where there is a bony part.

Another embodiment of the invention is a method as described above, wherein the proximal electrode (1) is placed in a drilled hole in said bony part, wherein said hole is drilled all the way through, or drilled partially through the bony part.

Another embodiment of the invention is a method as described above, wherein said proximal electrode (1) and output region (19) occupy the same said hole or occupy separately drilled holes.

Another embodiment of the invention is a method as described above, wherein the proximal electrode (1) and/or output region (19) are placed in the oval window.

Another embodiment of the invention is a method as described above, wherein the proximal electrode (1) and/or distal electrode (3) is pin-shaped and is implanted such that a longitudinal axis of the proximal electrode (1) and/or distal electrode (3) diverges from a longitudinal centreline of a cochlea (4) lumen.

Another embodiment of the invention is a method as described above, wherein the proximal electrode (1), vibrating surface (25) and/or distal electrode (3) is implanted such that it is flush or recessed with the inside wall of the inner ear (2).

Another embodiment of the invention is a method as described above, wherein the proximal electrode (1), vibrating surface (25) and/or distal electrode (3) is implanted such that it is flush or recessed with the inside wall of the lumen of the cochlea.

Another embodiment of the invention is a method as described above, wherein the distal electrode (3) is implanted such that the electrical impedance between it and the inner ear fluid at 1kHz is between 10 and 10 000 ohms.

Another embodiment of the invention is a method as described above, wherein the distal electrode (3) is implanted such that the electrical resistance between it and the proximal electrode (1) is between 10 and 10 000 ohms.

Another embodiment of the invention is a method as described above, wherein the distal electrode (3) is implanted such that the electrical impedance between it and the proximal electrode (1) at 1 kHz is between 10 and 10 000 ohms.

Another embodiment of the invention is a method as described above, further comprising the step of implanting a regulating unit (7), and connecting said electrodes (1, 3) and vibration generator (5) to said unit using one or more connecting electrical leads.

Another embodiment of the invention is a method as described above, wherein the proximal electrode, distal electrode, and vibration generator (5) are as defined above.

Another embodiment of the invention is a kit comprising the following components:
at least one proximal electrode (1),
at least one distal electrode (3),
at least one vibration generator (5),
one or more connecting electrical leads (8, 9, 10, 23, 24),
   and optionally one or more of the following:
a regulating unit (7),
surgical tools, and
instructions for use.

Another embodiment of the invention is a as described above, wherein said connecting electrical leads are disposed with connectors for connecting to the proximal electrode (1), distal electrode (3) and/or vibration generator (5).

Another embodiment of the invention is a as described above, wherein said where in the proximal electrode (1), distal electrode (3), and vibration generator (5) are as defined in above.

### DETAILED DESCRIPTION OF THE INVENTION

Unless defined otherwise, all technical and scientific terms used herein have the same meaning as is commonly understood by one of skill in the art. All publications referenced herein are incorporated by reference thereto. All United States patents and patent applications referenced herein are incorporated by reference herein in their entirety including the drawings.

The articles “a” and “an” are used herein to refer to one or to more than one, i.e. to at least one of the grammatical object of the article. By way of example, “an electrode” means one electrode or more than one electrode.

Throughout this application, the term “about” is used to indicate that a value includes the standard deviation of error for the device or method being employed to determine the value.

The recitation of numerical ranges by endpoints includes all integer numbers and, where appropriate, fractions subsumed within that range (e.g. 1 to 5 can include 1, 2, 3, 4 when referring to, for example, a number of electrodes, and can also include 1.5, 2, 2.75 and 3.80, when referring to, for example, a measurement).

The present invention relates to a method and device for improving hearing of a subject, based on the finding by the inventors that a significant improvement in hearing is achieved by:
electrically stimulating the auditory nerve 32 using two or more electrodes, none of which pass along a lumen of the cochlea (i.e. the scala tympani 42, scala vestibuli 40 or the scala media 41 of the cochlea), in combination with
mechanically stimulating the inner ear, especially the cochlea.

Because the electrodes do not pass along the scala tympani 42, scala vestibuli 40 or the scala media 41, the procedure is much less invasive than a traditional cochlea electrode, where the electrode enters and penetrates these areas.

In the present invention, a pair of electrodes can be attached anywhere near the cochlea, preferably outside the scala tympani 42, scala vestibuli 40 or the scala media 41 of the cochlea, to provide electrical stimulation of the cochlea. The electrodes in combination with mechanical (vibrational) stimulation of the inner ear, especially the cochlea improve hearing, while maintaining residual natural hearing in a less invasive surgical procedure.

The inventors have found that the electrodes can be placed in any configuration which provides electrical stimulation to the cochlea. In a preferred configuration, stimulation is achieved using a proximal electrode in physical (mechanical or actual) contact with a wall of the inner ear, and a distal (counter) electrode in electrical contact with the cochlea, more specifically the auditory nerve. Thus, a proximal electrode may be attached to a wall enclosing the inner ear, and a distal electrode may be attached or be sufficiently close to the auditory nerve to provide electrical contact.

Reference is made in the description below to the drawings which exemplify particular embodiments of the invention; they are not at all intended to be limiting. The skilled person may adapt the device and method, and substituent components and features according to the common practices of the person skilled in the art.

Device

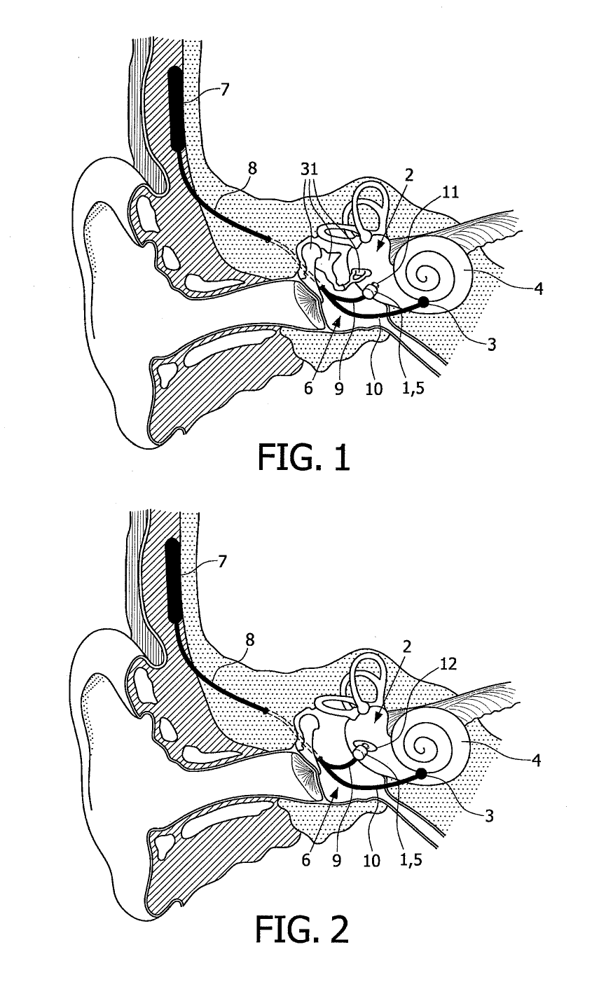

With reference to FIGS. 1 and 2, one embodiment of the present invention is an implantable device for improving hearing in a subject comprising:
a vibration generator 5 comprising an output region 19 configured to apply vibrational stimulation to the inner ear fluid,
a proximal electrode 1 configured for physical attachment to a wall enclosing the inner ear 2, at a location proximal to the output region 19, and
a separate distal electrode 3 configured to make electrical contact with an auditory nerve 32.

Proximal Electrode

The proximal electrode 1 is placed proximal to the output region 19 of the vibration generator 5 and is configured for physical attachment to a wall enclosing the inner ear 2.

The wall of the inner ear 2 refers to the tissues that enclose the inner ear 2 to form a fluid filled space. The inner ear 2 includes the cochlea with its scala vestibuli, scala typani and the various membranes and neural elements, the vestibulum and the semi-circular canals; such meaning is well understood in the art. The inner ear 2 may be regarded as the cavity bound by the cochlea 4 and the interface between the inner ear and the middle ear. Preferably, the proximal electrode 1 is configured for attachment to the outside of the wall enclosing the inner ear, i.e. on the non-fluid-filled side of the wall. Preferably, the proximal electrode is configured for attachment at the interface between the middle 6 and inner ear 2; the interface may include the promontorium. Preferably, the proximal electrode 1 is configured for attachment at the interface between the middle 6 and inner ear 2, where there is a bony part. Preferably, the proximal electrode 1 is configured for attachment at the interface between the middle 6 and inner ear 2, on the bony wall accessing the scala vestibuli 40 or the scala tympani 42. Preferably, the proximal electrode 1 is configured for attachment to an artificially drilled hole in the bony wall accessing the scala vestibuli (FIG. 1) or to the oval window 12 (FIG. 2). The proximal electrode 1 may attach either to the surface of the wall, to a small hole drilled partially through the wall, or to a small hole drilled all the way through the wall. The proximal electrode may be configured for attachment to a walled interface between the inner ear (2) and mastoid region. The proximal electrode may be configured for attachment to a walled interface between the inner ear (2) and mastoid region where there is a bony part.

The shape of a proximal electrode 1 can be any that permits implanting proximal to the vibration generator. Examples of shapes include, but are not limited to the following:
ball electrode configured for mounting onto or into the bony wall.
cylindrical pin configured for mounting onto or into the bony wall.
threaded pin configured for screwing into the bony wall.

The proximal 1 electrode may be provided with a measuring electrode for measuring the fluid or tissue voltage at the electrode interface. Such electrodes may be provided in a coaxial configuration whereby a tubular outer member provides the stimulation and a central pin measures the fluid or tissue voltage. The tubular outer member may have a smooth surface or may be threaded for screwing into a bony wall. An alternative configuration of the measuring electrode is where it is provided in the metal wall of the vibration generator, for example, as a pin, but electrically insulated therefrom; the metal wall of the vibration generator acts as the proximal electrode and stimulates the acoustic nerve while the pin is used to measure the fluid or tissue voltage at the electrode interface. Another alternative of the measuring electrode is where it is provided as part of the vibration generator as a coaxial arrangement with the proximal electrode; a coaxial electrode embedded in the metal wall of vibration generator, but electrically insulated from it. The outer coaxial sleeve is electrically driven to stimulate the acoustic nerve, and where the central pin is used to measure the fluid or tissue voltage right at the electrode interface.

Such a measurement can be part of a control loop that may automatically adjust the stimulation current on the proximal electrode to obtain a desired neural response and/or be used to control the vibrational stimulation. One embodiment of the invention, therefore, is a device as described herein, wherein the regulating unit 7 is configured to use measurements from a measuring electrode for closed-loop control of the electrical and/or vibrational stimulation.

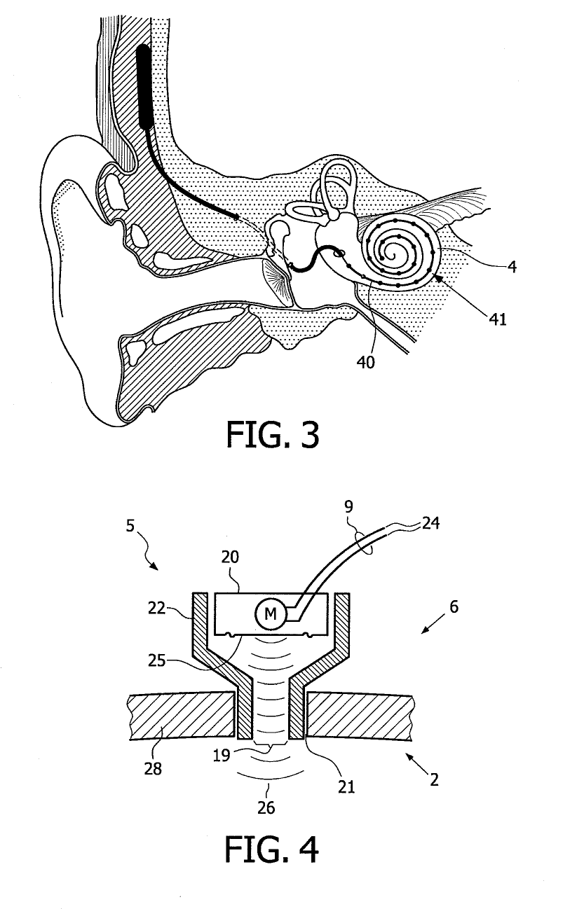

According to one embodiment of the invention, the proximal electrode 1 penetrates a lumen of the cochlea 4 (e.g. the scala tympani 42, scala vestibuli 40 or the scala media 41) and contacts the fluid of the lumen. Where the electrode is pin-shaped, a longitudinal axis of the electrode may be divergent from a longitudinal centreline of a cochlea 4 lumen. In other words, a pin-shaped electrode may not lie along the passage of a lumen of the cochlea 4. The longitudinal axis and centreline may preferably be about perpendicular. This configuration is distinct from the prior art (e.g. FIG. 3) where an electrode 40 typically runs along the length of the passage of the scala tympani 42, scala vestibuli 40 or the scala media 41 such that the longitudinal axis of the electrode 40 and the longitudinal centreline of a cochlea lumen 41 essentially coincide or are parallel.

Where the proximal electrode 1 penetrates a lumen of the cochlea 4 (e.g. the scala vestubuli 40, scala media 41 or scala tympani 42) and contacts the fluid therein, the electrode may or may not extend into a lumen. Where it does not, the electrode may be flush with the inside wall of a lumen, or recessed with the inside wall. Where it does, it may only extend by amount so as not to damage the fragile basilar and Reissner membranes, the spiral organ, the organ of Corti, or the sensory hair cells of the cochlea. According to one embodiment of the invention, the proximal electrode 1 extends into a lumen of the cochlea, by a distance less than or equal to 2 mm, 1.8 mm, 1.6 mm, 1.4 mm, 1.2 mm, 1 mm, 0.8 mm, 0.6 mm, 0.4 mm, 0.2 mm, 0.1 mm, 0.08 mm, 0.06 mm, 0.04 mm, 0.02 mm, or by an amount in the range between any two of the aforementioned values. Preferably the distance is between 0.1 and 0.5 mm.

In one embodiment of the invention, the proximal electrode is a short intracochlear electrode that extends into the a lumen of the cochlea 4, without damage to the fragile basilar and Reissner membranes, the spiral organ, the organ of Corti, or the sensory cells (hair cells). According to one aspect, an intracochlear electrode extends into a lumen of the cochlea 4 by a distance less than or equal to 15 mm, 14 mm, 12 mm, 10 mm, 8 mm, 6 mm, 4 mm, 3 mm, or by an amount in the range between any two of the aforementioned values. Preferably the distance is between 3 and 15 mm.

The proximal electrode 1 is configured for physical attachment to a wall enclosing the inner ear 2. This means it is implantable. As such, it should fulfil the requirements for an implant such as biocompatibility, stability, and be of suitable shape and size for attachment. The proximal electrode 1 may be made from any suitable biocompatible conducting material such as surgical steels, or platinum, iridium, titanium, gold, silver, nickel, cobalt, tantalum, molybdenum, or their biocompatible alloys. The skilled person may employ material as known in the prior art, for example as described in Venugopalan R. and R. Ideker, “Bioelectrodes,” in Biomaterial Science—An Introduction To Materials in Medicine, Eds. B. D. Ratner, A. S. Hoffman, F. J. Schoen and J. E. Lemons, Elsevier Academic Press, ISBN 0-12-582463-7, pp. 648-657. The proximal electrode may be coated with a substance that lowers its DC and/or AC impedance. Examples of suitable impedance lowering substances include porous platinum coating, titanium nitride coating with or without carbon, iridium coating, iridium oxide coating, titanium nitride coating with iridium oxide, tantalum-based coatings. The number of proximal electrodes may be 1, 2, 3, 4, 5, 6, 7, 8, 9, 10, or more. The number of proximal electrodes may equal the number of distal electrodes.

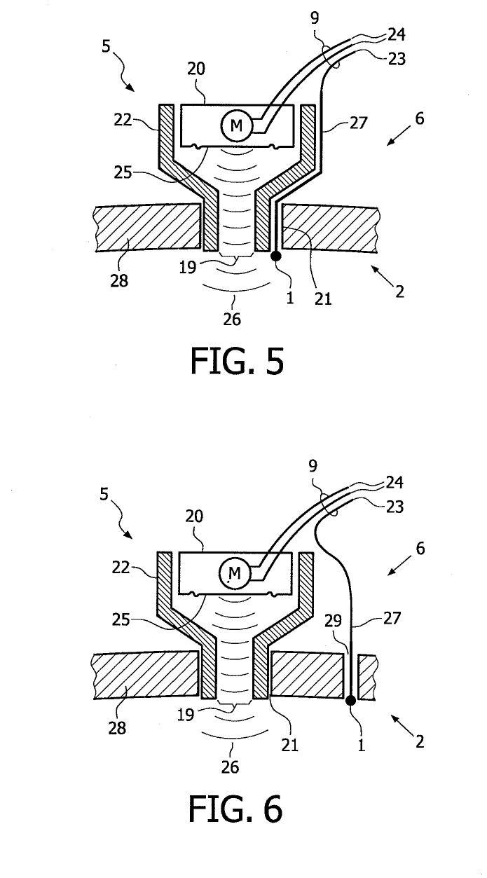

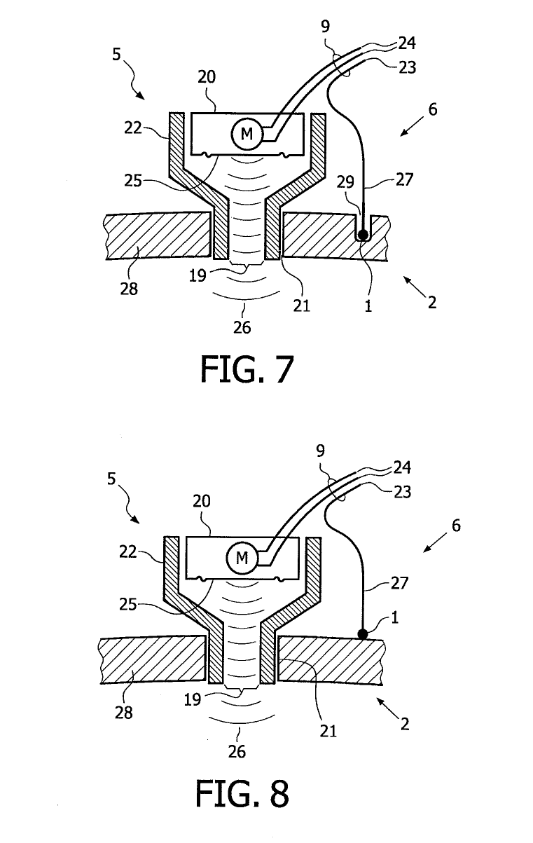

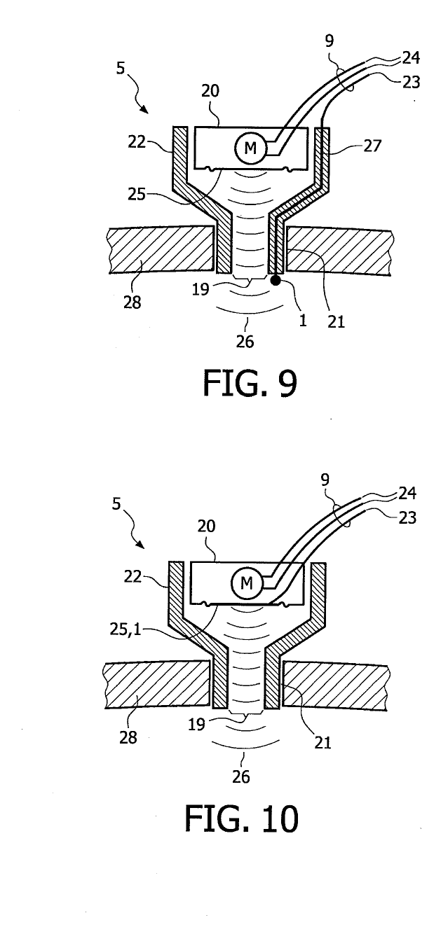

According to one aspect of the invention, a proximal electrode 1 is configured to attach to a wall enclosing the inner ear 2, in close proximity to the output region 19 of the vibration generator 5. This configuration means the output region 19 and the proximal electrode 1 are close together, so making implantation easier. The proximal electrode 1 may be attached to the surface of the wall, adjacent to the output region 19; this embodiment is seen, for example, in FIGS. 8, 12 and 16. The output region 19 and proximal electrode 1 may share the same hole; this embodiment is seen, for example, in FIGS. 5, 11 and 15. The proximal electrode 1 may be disposed in a hole 29, adjacent to the output region 19, and contact the inner ear fluid; this embodiment is seen, for example, in FIGS. 6, 13, 17, 21 and 24 to 30 where the proximal electrode 1 is disposed in a separate small hole 21. The proximal electrode 1 may be disposed in a hole 29, adjacent to the output region 19, which hole only partially penetrates the interface; this embodiment is seen, for example, in FIG. 7, where the proximal electrode 1 is disposed in a separate small hole 21. Alternatively, the proximal electrode 1 may be comprised in the vibration generator 5; this embodiment is seen, for example, in FIG. 9 (where it is part of the frame 22), and FIGS. 10, 14 and 18 (where it is part of the output region 19, particularly the vibrating surface 25). According to one aspect of the invention, the output region 19 and the proximal electrode 1 are less than or equal to 10 mm, 9.5 mm, 9.0 mm, 8.5 mm, 8.0 mm, 7.5 mm, 7.0 mm, 6.5 mm, 6.0 mm, 5.5 mm, 5.0 mm, 4.5 mm, 4.0 mm, 3.5 mm, 3.0 mm, 2.5 mm, 2.0 mm, 1.0 mm, 0.1 mm, 0.01 mm apart, or a distance apart that is in the range between any two of the aforementioned values. Preferably the distance is between 0.01 and 5.0 mm.

Distal Electrode

The distal electrode 3 is separate from the proximal electrode 1, and is placed apart therefrom. The distal electrode 3 is configured to make electrical contact with the auditory nerve 32. It may or may not be in physical (mechanical) contact with the auditory nerve 32 to achieve this. Where it is in physical contact with the auditory nerve 32, it may be attached thereto.

Where the distal electrode 3 is not in physical contact with the auditory nerve 32, it may be attached to a wall enclosing the cochlea 4. In which case, the distal electrode 3 is preferably configured for attachment to the outside of the wall enclosing the cochlea 4, i.e. on the non-fluid-filled side of the wall. The distal electrode 3 may attach either to the surface of the wall, to a small hole drilled partially through the wall, or through a small hole drilled all the way through the wall.

According to one embodiment of the invention, the distal electrode 3 is configured for attachment at the interface between the middle 6 and inner ear 2. The distal electrode 3 may be configured for attachment at the interface between the middle 6 and inner ear 2, where there is a bony part; the interface may include the promontorium. The distal electrode 3 may be configured for attachment at the interface between the middle 6 and inner ear 2, on the bony wall accessing the scala vestibuli or the scala timpani. The distal electrode 3 may be configured for attachment to an artificially drilled hole in the bony wall accessing the scala vestibuli or to the oval window. The distal electrode 3 may be configured for attachment to a walled interface between the inner ear 2 and mastoid region. The distal electrode 3 may be configured for attachment to a walled interface between the inner ear 2 and mastoid region where there is a bony part.

According to one embodiment of the invention, the distal electrode 3 penetrates a lumen of the cochlea 4 (e.g. the scala tympani 42, scala vestibuli 40 or the scala media 41) and contacts the fluid of the lumen. Where the electrode is pin-shaped, a longitudinal axis of the electrode may be divergent from a longitudinal centreline of a cochlea 4 lumen. In other words, a pin-shaped distal electrode 3 may not lie along a passage of a lumen of the cochlea 4. The longitudinal axis and centreline may preferably be about perpendicular. This configuration is distinct from the prior art (e.g. FIG. 3) where an electrode 40 typically runs along the length of the passage of the scala tympani 42, scala vestibuli 40 or the scala media 41 such that a longitudinal axis of the electrode 40 and the longitudinal centreline of the cochlea lumen 41 essentially coincide or are parallel.

Where the distal electrode 3 penetrates a lumen of the cochlea 4 and contacts the fluid of the lumen, the electrode may or may not extend into the lumen. Where it does not, the electrode may be flush with the inside wall of the lumen, or recessed with the inside wall. Where it does, it may only extend by amount not to damage the fragile basilar and Reissner membranes, the spiral organ, the organ of Corti, or the sensory cells (hair cells) inside the cochlea. According to one embodiment of the invention, the distal electrode 3 extends into the lumen by a distance less than or equal to 2 mm, 1.8 mm, 1.6 mm, 1.4 mm, 1.2 mm, 1 mm, 0.8 mm, 0.6 mm, 0.4 mm, 0.2 mm, 0.1 mm, 0.08 mm, 0.06 mm, 0.04 mm, 0.02 mm, or by an amount in the range between any two of the aforementioned values. Preferably the distance is between 0.1 and 0.5 mm.

Where the distal electrode 3 is not in physical contact with the auditory nerve 32, it is sufficiently close thereto to retain electrical contact with the auditory nerve 32 or the neural elements inside the cochlea. This means the auditory nerve or the neural elements inside the cochlea can be electrically stimulated by passing electrical current between said distal electrode 3 and proximal electrode 1. This may also mean that the electrical impedance between the distal electrode 3 and the inner ear fluid at 1 kHz may be less than or equal to 100 000 ohms, 80 000 ohms, 60 000 ohms, 40 000 ohms, 20 000 ohms, 10 000 ohms, 8 000 ohms, 5 000 ohms, 2 000 ohms, 1000 ohms, 800 ohms, 600 ohms, 400 ohms, 200 ohms, 100 ohms, 50 ohms, or a value in the range between any two of the aforementioned values. Preferably the impedance is between 10 and 10 000 ohms.

According to one aspect of the invention, the distal electrode 3 is positioned such that the electrical resistance between it and the proximal electrode 1 is less than or equal to 100 000 ohms, 80 000 ohms, 60 000 ohms, 40 000 ohms, 20 000 ohms, 10 000 ohms, 8 000 ohms, 5 000 ohms, 2 000 ohms, 1000 ohms, 800 ohms, 600 ohms, 400 ohms, 200 ohms, 100 ohms, 50 ohms, or a value in the range between any two of the aforementioned values. Preferably the resistance is between 10 and 10 000 ohms.

According to one aspect of the invention, the distal electrode 3 is placed such that the electrical impedance between it and the proximal electrode 1 at 1 kHz is less than or equal to 100 000 ohms, 80 000 ohms, 60 000 ohms, 40 000 ohms, 20 000 ohms, 10 000 ohms, 8 000 ohms, 5 000 ohms, 2 000 ohms, 1000 ohms, 800 ohms, 600 ohms, 400 ohms, 200 ohms, 100 ohms, 50 ohms, or a value in the range between any two of the aforementioned values. Preferably the impedance is between 10 and 10 000 ohms.

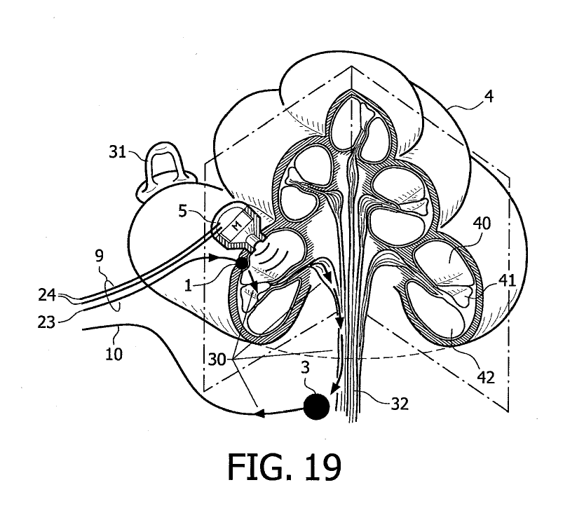

The circuit formed by the proximal electrode 1 and distal electrode 3 is shown in FIG. 19. In this figure, the distal electrode 3 is in proximity of the auditory nerve 32, and the proximal electrode 1 attached to the wall of the cochlea 4. Depending on the polarity of the signal provided by the wires 10, 23, current may flow 30 from the proximal electrode 1 to the distal electrode 3 along the arrows indicated. The polarity of the signal may equally change, and the current flow in the opposite direction (not shown).

According to one embodiment of the invention, the distal electrode 3 is configured for attachment in the vicinity of the inner ear 2. As mentioned above, it may be in contact with the cochlea 4, on the non-fluid-filled side of the wall. It may make contact with the auditory nerve. For instance, it may be implanted in a hole accessing the singular nerve (posterior ampullary nerve) canal that passes vestibular nerve fibres to the auditory brain stem, providing a low-impedance connection to the auditory nerve. Alternatively, the distal electrode 3 may be remote from the cochlea 4. According to one aspect of the invention, it may be disposed within an implanted regulating unit 7. For example, it may be disposed as an electrically conductive patch on the exterior housing of the regulating unit 7. Alternatively, the distal electrode may be the casing itself of the regulating unit 7.

The distal electrode 3 is implantable. As such, it should fulfil the requirements for an implant such as biocompatibility, stability, and be of suitable shape and size for attachment. The distal electrode 3 may be made from any suitable biocompatible conducting material such as surgical steels, or platinum, iridium, titanium, gold, silver, nickel, cobalt, tantalum, molybdenum, or their biocompatible alloys. The distal electrode may be coated to lower its DC and/or AC impedance; examples of suitable coatings include porous platinum, titanium nitride with or without carbon, iridium, iridium oxide, titanium nitride with iridium oxide, or tantalum-based coatings. The number of distal electrodes may be 1, 2, 3, 4, 5, 6, 7, 8, 9, 10 or more. The number of distal electrodes may equal the number of proximal electrodes.

The shape of a distal electrode 3 can be any that permits implanting to make electrical contact with an auditory nerve 32. Examples of shapes include, but are not limited to the following:
ball electrode configured for mounting onto or into the bony wall.
cylindrical pin configured for mounting onto or into the bony wall.
threaded pin configured for screwing into the bony wall.

The distal electrode 3 may be provided with a measuring electrode for measuring the tissue voltage at the electrode interface. Such electrodes may be provided in a coaxial configuration whereby a tubular outer member provides the stimulation and an central pin measures the tissue or fluid voltage. The tubular outer member may have a smooth surface or may be threaded for screwing into a bony wall. An alternative configuration of the measuring electrode is where it is provided in the metal wall of the vibration generator, for example, as a pin, but electrically insulated therefrom; the metal wall of the vibration generator acts as the distal electrode and stimulates the acoustic nerve while the pin is used to measure the tissue or fluid voltage at the electrode interface. Another alternative of the measuring electrode is where it is provided as part of the vibration generator as a coaxial arrangement with the distal electrode; a coaxial electrode embedded in the metal wall of vibration generator, but electrically insulated from it. The outer coaxial sleeve is electrically driven to stimulate the acoustic nerve, and where the central pin is used to measure the tissue or fluid voltage right at the electrode interface.

Vibration Generator

The vibration generator 5 according to the invention comprises a vibrating output region 19 configured to apply vibrational stimulation to the inner ear fluid.

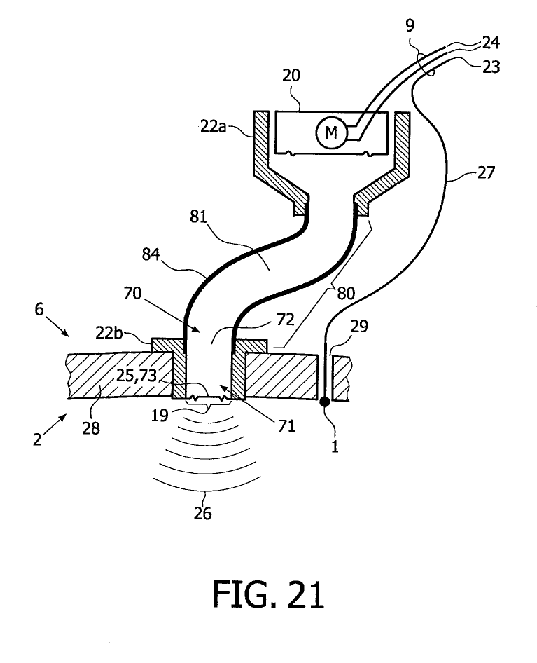

FIGS. 4 and 21 show examples of a vibration generator 5 in situ. Typically a vibration generator 5 comprises a frame 22, optionally formed from two subframes 22a, 22b, which frame is configured for attachment to a wall of the middle ear. In FIG. 4, the frame 22 is formed from a single elements and is attached to a hole 21 in the interface 28 between inner ear 2 and the middle ear 6. In FIG. 21, the frame 22 comprises a first remote sub-frame 22a that is attached to a bony part of the middle ear or mastoid region and a second sub-frame 22b attached to a hole 21 in the interface 28 between inner ear 2 and the middle ear 6. Vibrational stimulation is generated by an electromechanical actuator 20 that is held in place by the frame 22. Co-operatively connected (e.g. rigidly, flexibly or semi-flexibly) to the electromechanical actuator 20 is a vibrating surface 25 which provides vibrational energy. As elaborated below, the vibrating surface 25 may be formed from a membrane, a pin or plate-like structure, or from any suitable shaped element. Frame 22 is configured to position the vibrating surface 25 so as to direct the vibrational energy therefrom to the output region 19. Frame 22 is also configured to position the output region 19 to provide said vibrational stimulation to the inner ear fluid. The frame may comprise a housing for the electromechanical actuator 20; such housing may protect the actuator from exposure to fluids present in the middle ear 6.

An electrical lead 9 with lead wires 24 generally connects the electromechanical actuator 20 to a regulating unit 7. The lead wires 24 carry processed sound information to the vibration generator 5. The sound information may be full audio spectrum sound. Alternatively, the sound information may be processed, for example, low-frequency filtered, high-frequency filtered or multi-band processed. A vibrating surface 25 of the electromechanical actuator 20 vibrates according to the signal on the lead wires, and causes mechanical vibrations 26 that propagate in the inner ear fluid. The mechanical vibration generator 5 thus comprises an electromechanical actuator 20 that converts the electrical signals transmitted by the lead wires 24 to mechanical vibrations 26, which are coupled to the inner ear fluid ultimately by the vibrating surface 25.

According to one aspect of the invention, a frame 22 holds the electromechanical actuator 20 and also formed to provide an output region 19 that may be an aperture in the frame 22 through which vibrational energy is directed. The frame 22 may be composed of a single element; this is shown, for example, in FIGS. 4 to 10, where the frame encloses the electromechanical actuator 20, and forms an aperture that provides an output region 19.

The frame 22 of the vibration generator 5 may be configured for physical attachment to a wall enclosing the middle ear 6. The wall is usually solid tissue (e.g. bone). Preferably, the frame 22 of vibration generator 5 is configured for attachment to the outside of the wall enclosing the inner ear 2, i.e. on the non-fluid-filled side of the wall; this configuration is shown, for example, in FIGS. 4 to 14. Preferably, the frame 22 is configured for attachment at the interface between the middle 6 and inner ear 2; the interface may include the promontorium. Preferably, the frame 22 is configured for attachment at the interface between the middle 6 and inner ear 2, where there is a bony part. Preferably, the frame 22 is configured for attachment at the interface between the middle 6 and inner ear 2, on the bony wall accessing the scala vestibuli 40 or the scala tympani 42. Preferably, the frame 22 is configured for attachment to an artificially drilled hole in the bony wall accessing the scala vestibuli (FIG. 1), or to the oval window 12 (FIG. 2). The frame 22 may attach either to the surface of the wall, to a small hole drilled partially through the wall, or to a small hole drilled all the way through the wall.

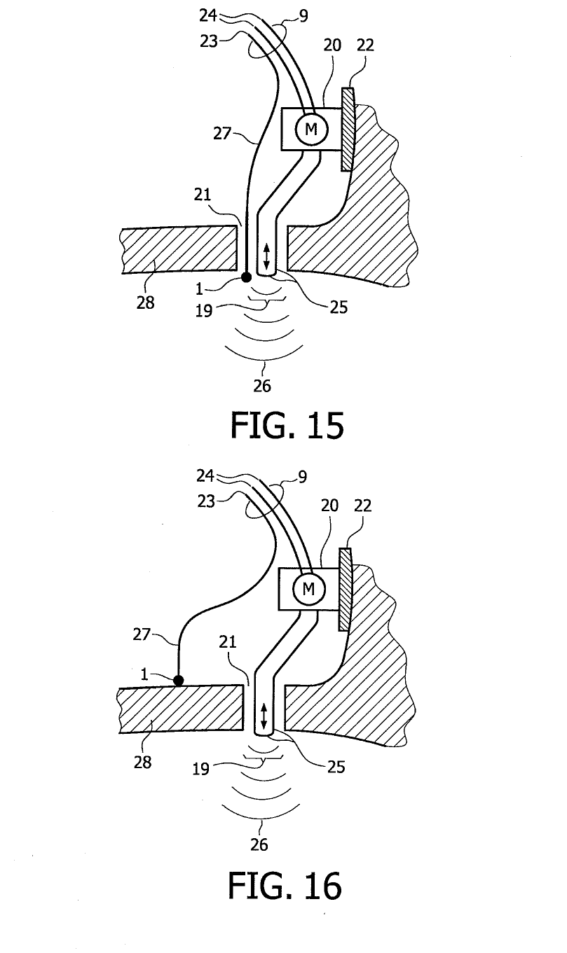

According to another embodiment of the invention, the frame 22 is configured for attachment to a wall enclosing the middle ear 6, which wall is not an interface 28 between the middle 6 and inner ear 2. This is exemplified in FIGS. 15 to 18, where the wall is adjacent to said interface 28.

According to yet another embodiment of the invention, the frame 22 is configured for embedding in a cavity machined in a bony wall enclosing the inner ear, e.g. in the mastoid or temporal bone.

According to yet another embodiment of the invention, the frame 22 is configured for attachment at the interface between the inner ear 2 and the mastoid region. According to yet another embodiment of the invention, the frame 22 is configured for attachment at the interface between the inner ear 2 and the mastoid region where there is a bony part. According to yet another embodiment of the invention, the frame 22 is configured for embedding in a bony wall between the vestibule and the mastoid region. The mastoid region contains mastoid cells that are air-filled pockets in the mastoid process that connect to the middle ear. In implanting the frame 22, the mastoid cells are removed when a skilled practitioner e.g. surgeon carves out the mastoid to create access to the vestibulum. This surgical procedure is called a mastoidectomy. We have recently found that the inner-ear vestibule can be accessed surgically from behind the ear via the mastoid, so allowing convenient implantation.

The frame 22 is implantable. As such, it should fulfil the requirements for an implant such as being form from or coated with a biocompatible and stable material, and be of suitable shape and size for insertion and placement. The parts of the frame 22 in contact with tissue and/or fluid may be made from any suitable biocompatible material, for example, surgical steels, or platinum, iridium, titanium, gold, silver, nickel, cobalt, tantalum, molybdenum, or their biocompatible alloys.

Vibration Generator—Subframes

According to another aspect of the invention, the frame 22 comprises at least two distinct parts; a remote, first sub-frame 22a that supports and holds in place the electromechanical actuator 20 and a second sub-frame 22b configured for attachment at the interface between the middle 6 and inner ear 2, and which provides the output region 19. The first subframe 22a is configured to position the electromechanical actuator 20 so as to direct the vibrational energy therefrom to the output region 19 present in the second sub-frame 22b. Vibration energy from the electromechanical actuator 20 is directed to the output region 19 via a vibrational-energy conducting element 80, which may be, for example, a liquid filled tube, a cable connection, or a rod link, which conducting elements are elaborated below. The two-part frame allows the electromechanical actuator 20 advantageously to be positioned remote from the output region 19, for example, in circumstances where the physiology of the subject does not allow the implant of a single-frame vibration generator 5.

Vibration Generator—First Sub-Frame

The first sub-frame 22a comprises a housing for the electromechanical actuator 20; such housing may protect the actuator from exposure to fluids present in the middle ear 6 or elsewhere. According to one aspect of the invention, the first sub-frame 22a of the vibration generator 5 is configured for physical attachment in the middle ear cavity. Preferably, the first sub-frame 22a of the vibration generator 5 is configured for physical attachment to a supporting wall enclosing the middle ear 6 as shown, for example, in FIGS. 21, 24, 25, 26, 27, 28, 29 and 30. The wall is usually solid tissue (e.g. a bony wall of the middle ear cavity).

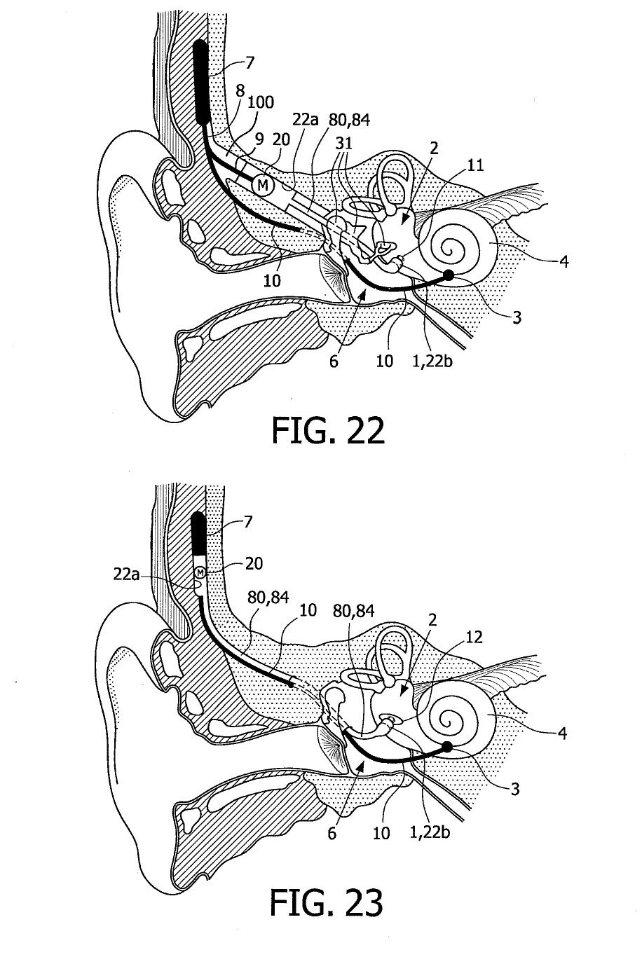

According to another aspect of the invention, the first sub-frame 22a of the vibration generator 5 is configured for placement in a cavity 100 as shown, for example, in FIG. 22 where it is implanted in the mastoid region. According to the illustrated embodiment, a tube 84 carries a hydraulic connection to the output region of the second sub-frame 22b.

According to yet another embodiment of the invention, the first sub-frame 22a is configured for embedding in a cavity machined in a bony wall enclosing the inner ear, e.g. in the mastoid or temporal bone.

According to yet another embodiment of the invention, the first sub-frame 22a is configured for attachment to a bony wall of a cavity created in the mastoid region.

According to yet another embodiment of the invention, the first sub-frame 22a is configured for embedding in a bony wall between the vestibule and the mastoid region. The mastoid region contains mastoid cells that are air-filled pockets in the mastoid process that connect to the middle ear. In implanting the first sub-frame 22a, the mastoid cells are removed when a skilled practitioner e.g. surgeon carves out the mastoid to create access to the vestibulum. This surgical procedure is called a mastoidectomy. As already mentioned, we have found that the inner-ear vestibule can be accessed surgically from behind the ear via the mastoid, so allowing convenient implantation. According to another yet another aspect of the invention, the first sub-frame 22a of the vibration generator 5 is incorporated within the housing of the regulating unit 7, as shown, for example, in FIG. 23. According to the illustrated embodiment, a tube 80 carries a hydraulic connection to the output region of the second sub-frame 22b.

The first sub-frame 22a is implantable. As such, it should fulfil the requirements for an implant such as being form from or coated with a biocompatible and stable material, and be of suitable shape and size for insertion and placement. The parts of the first sub-frame 22a in contact with tissue and/or fluid may be made from any suitable biocompatible material, for example, surgical steels, or platinum, iridium, titanium, gold, silver, nickel, cobalt, tantalum, molybdenum, or their biocompatible alloys.

Vibration Generator—Second Sub-Frame

The second sub-frame 22b may be configured for attachment to a walled interface between the middle 6 and inner ear 2; the interface may include the promontorium. Preferably, the second sub-frame 22b is configured for attachment at the interface between the middle 6 and inner ear 2, where there is a bony part. The second sub-frame 22b of the vibration generator 5 may be configured for physical attachment to a walled interface between the between the middle 6 and inner ear 2. Preferably, the second sub-frame 22b is configured for attachment at the interface between the middle 6 and inner ear 2, on the bony wall accessing the scala vestibuli 40 or the scala tympani 42. Preferably, the second sub-frame 22b may access the scala vestibule 40, the scala tympani 42, or the vestibulum. Preferably, the second sub-frame 22b is configured for attachment to an artificially drilled hole in the bony wall accessing the scala vestibuli, or to the oval window 12. The second sub-frame 22b may attach either to the surface of the wall, to a small hole drilled partially through the wall, or to a small hole drilled all the way through the wall. The second sub-frame 22b may be configured for attachment to a walled interface between the inner ear 2 and the mastoid region. The second sub-frame 22b may be configured for attachment to a walled interface between the inner ear 2 and the mastoid region where there is a bony part. Preferably, the second sub-frame 22b is configured for attachment to a bony wall of the middle ear cavity, or for attachment to a bony wall in the mastoid region, or for embedment in a cavity created in the mastoid region.

As mentioned above, the proximal electrode may be incorporated into the vibration generator 5; where the vibration generator 5 is formed from a multi-element-frame as described above, the proximal electrode 1 may be comprised in the second-sub frame 22b or in the vibrating surface 25.

The second sub-frame 22b is implantable. As such, it should fulfil the requirements for an implant such as being form from or coated with a biocompatible and stable material, and be of suitable shape and size for insertion and placement. The parts of the second sub-frame 22b in contact with tissue and/or fluid may be made from any suitable biocompatible material, for example, surgical steels, or platinum, iridium, titanium, gold, silver, nickel, cobalt, tantalum, molybdenum, or their biocompatible alloys.

The second sub-frame 22b is preferably disposed with a passage 72, essentially cylindrical in shape, having a receiving end 70 to receive vibrational energy from the conducting element 80, and a transmitting end 71 where vibrational energy is directed towards the inner ear fluid. The passage 72 may be at least partly linear, though other shapes are envisaged including curved or angular. A region towards or at the transmitting end 71 may be disposed with the vibrating surface 25 (e.g. membrane, a plate, piston) that is able to vibrate responsive to vibrations generated by the electromechanical actuator 20 and which surface is in physical contact with the inner ear fluid. FIGS. 21, 24, 25, 27, 28, 29 and 30 depict embodiments where the transmitting end 71 of the passage 72 is provided with a vibrating surface 25.

In FIG. 21, the vibrating surface 25 is a flexible or flexibly suspended membrane 73 which seals the transmitting end 71 of the passage 72 and is hydraulically moved forward and backwards by fluid 81 in a tube 84 that forms the conducting element 80. By sealing, it is meant that a water-impermeable barrier is formed. The membrane 73 is preferably made from a water impermeable material. The material may be flexible i.e. will change shape in response to the applied hydraulic pressure. Alternatively, it may be rigid, but connected to the passage 72 by a flexible suspension, and the rigid membrane 73 moves without changing shape in response to the applied hydraulic pressure.

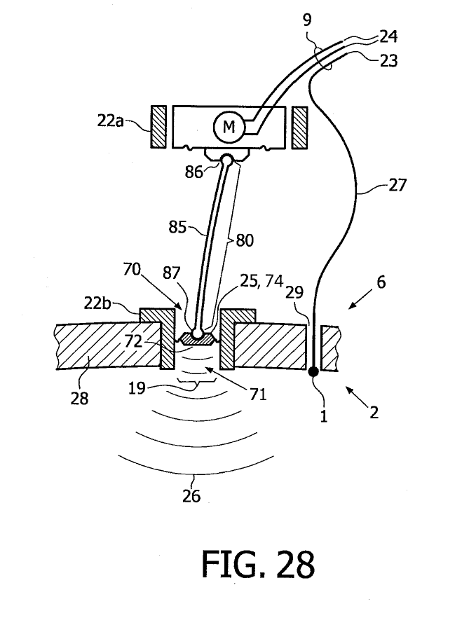

In FIGS. 28 to 30, the vibrating surface 25 is a formed from a rigid plate 74 which is attached to the passage 72 of the second sub-frame 22b by a flexible suspension. Owing to the suspension, the plate 74 is able to vibrate responsive to vibrations generated by the electromechanical actuator 20 without substantial shape change. The plate 74 is moved forward and backwards by means of a mechanical link such as a rod, a telescopic link or hinged link as elaborated below. Because hydraulic pressure is preferably not used, it is not always necessary that the plate 74 seals the passage, but sealing is not excluded either, for example, to prevent leakage of inner ear fluid through the passage 72 of the second sub-frame 22b.

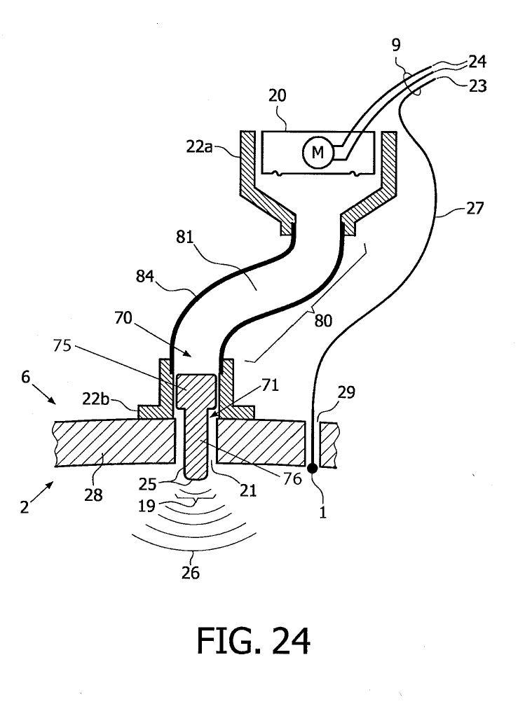

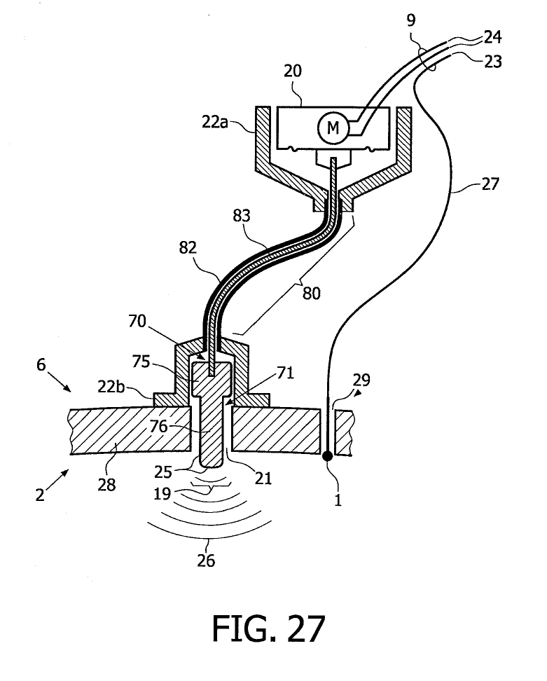

FIGS. 24 and 27 depict an embodiment where the vibrating surface 25 is a formed from a sliding piston 75 that can move linearly along the passage 72. The piston 75 may be extended with a pin 76. The pin may protrude from the transmitting end 71 of the passage 72. Movements of the piston 75 may be hydraulically controlled (FIG. 24) in which case the piston 75 forms a seal against the passage 72 wall. The seal may be water-tight or may have a leakage rate that is not detrimental to the application of hydraulic pressure. It is noted that a water tight seal is not essential for proper functioning of the piston. Limited fluid leakage around the piston does not affect audio transfer, and may serve to equalize the static pressure in the hydraulic tube with the inner ear pressure. A water tight seal may be employed, for example, in circumstances when the hydraulic fluid is other than inner ear fluid, and mixing of the respective fluids is to be avoided. Alternatively, the piston 75 may be controlled by a flexible cable 83 (FIG. 27), in which case a water-tight seal is not essential, but not excluded. A water tight seal may be included in the instance when a lubricant is used between the cable jacket 82 and the cable 83 to ensure smooth operation, and the lubricant should not mix with the inner ear fluid. According to one aspect of the invention, the inner ear fluid is used as a lubricant, in which case a perfectly sealing piston is not required. A watertight seal may then reside closer to the receiving end 70 of the passage 72 to avoid loss of the inner ear fluid. The vibrating surface 25 may be formed by the part of the pin facing the transmission end 71 of the passage 72; it may be formed by a protrusion of the pin from the second sub-frame 22b.

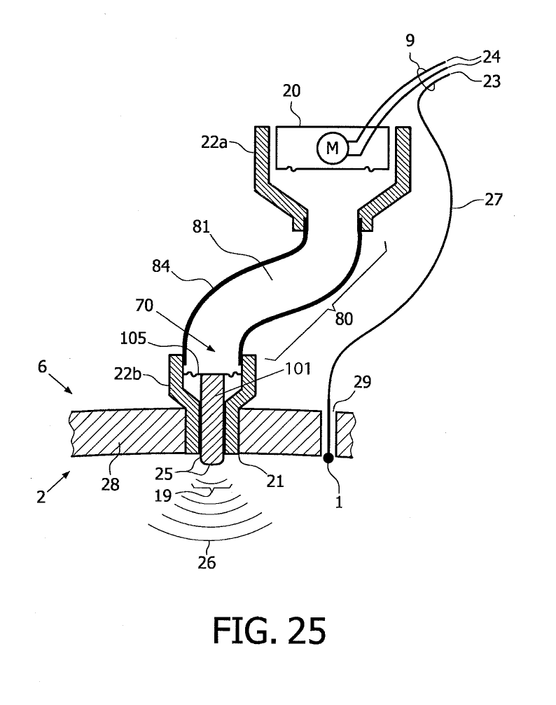

FIG. 25 depicts the embodiment where the passage 72 is sealed with a flexibly suspended membrane 105. The flexible suspension, as mentioned above, allows a rigid membrane to move without changing shape in response to the applied hydraulic pressure. Rigidly attached to said membrane is a pin 101 that moves in concert with the membrane 100, and which protrudes from the transmitting end 71 of the passage 72. The pin 101 is able to vibrate responsive to vibrations generated by the electromechanical actuator 20 via hydraulic coupling. The vibrating surface 25 is formed by the protrusion of the pin 101 from the second sub-frame 22b.

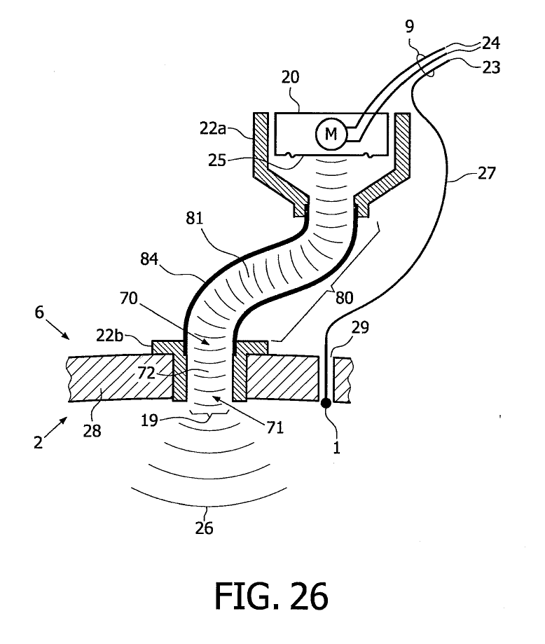

It is also within the scope of the invention that the passage 72 is devoid of a vibrating surface 25, such as the membrane 105, pin 101 or piston 75; this is depicted in FIG. 26. In this instance, the vibrating surface 25 is found close to the electromechanical actuator 20, and vibrations therefrom are propagated through the tube 84 and leave the transmitting end 71 of the passage 72 where they physically stimulate the inner ear fluid.

Conducting Elements

As already mentioned above, vibration energy generated by the electromechanical actuator 20 present in the first sub-frame 22a is carried to the output region 19 present in the second sub-frame 22b via a conducting element 80, which may be, for example, a fluid containing tube, a cable connection, or a rod link; these conducting elements are elaborated below.

Fluid-Containing Tube

According to one aspect of the invention, the conducting element 80 is a tube 84 adapted to contain a fluid, which carries vibrational energy via the non-compressible liquid medium 81. Such aspect is depicted in FIGS. 21, 22, 23, 24, 25, and 26. The tube 84 is attached at one end to an opening in the first sub-frame 22a and at the other end to an opening in the second sub-frame 22b. The interior of the tube 84 is in fluid connection with the electromechanical actuator 20 of the first sub-frame 22a, and with at least part of the passage 72 present in the second sub-frame 22b. The tube 84 may be filled with a fluid that is a non-compressible liquid or gel 81. The vibrational motions are transferred to the vibrating surface 25 or to the output region 19 which is in vibrational contact with the inner ear fluid. The tube 84 is sufficiently flexible or malleable so that it can be shaped during surgery to adapt it to the anatomy of the specific patient.

The tube 84 should fulfil the requirements for an implant such as being formed from or coated with a biocompatible and stable material, and be of suitable shape and size for insertion and placement. The tube 84 is preferably made from a flexible or malleable, non-expandable, material. The tube 84 is preferably water impermeable to the extent that it is able to retain fluid under hydraulic pressure, without significant leakage through the tube detrimental to hydraulic transmission. The parts of the tube 84 in contact with tissue and/or fluid may be made from any suitable biocompatible material having these properties, for example, PTFE tubing, polypropylene tubing, braid-reinforced silicone or polyimide tubing, polyketone (e.g. polyetheretherketone or PEEK™) tubing, or poly-ethylene tubing.

Cable Link

According to another aspect of the invention, the conducting element 80 is a flexible cable link, comprising a flexible cable 83 covered by a flexible sleeve 82, which cable 83 is configured to move within the sleeve 82, for example a rotation or a displacement, while maintaining a coaxial relation with the sleeve 82. Such aspect is depicted in FIG. 27. The sleeve 82 is mechanically attached at one end to the first sub-frame 22a, and at the other end to the second sub-frame 22b, preferably such that the interior of the sleeve 82 forms a chamber with both the electromechanical actuator 20 and the rear side of the vibrating surface 25. The cable 83 passes through the sleeve 82, mechanically joining the electromechanical actuator 20 of the first sub-frame 22a, with the vibrating surface 25, more particularly, a pin 75, present in the second sub-frame 22b.

The cable 83 and sleeve 82 should fulfil the requirements for an implant such as being formed from or coated with a biocompatible and stable material, and be of suitable shape and size for insertion and placement. The cable 83 is preferably made from a flexible, non-stretchable material. The parts of the cable 83 in contact with tissue and/or fluid may be made from any suitable biocompatible material having these properties stainless steel, stainless steel alloy, titanium, nickel or any suitable material. The sleeve 82 is preferably made from a flexible, non-compressible material. The parts of the sleeve 82 in contact with tissue and/or fluid may be made from any suitable biocompatible material having these properties stainless steel, stainless steel alloy, titanium, nickel, PTFE, polypropylene, silicone, polyimide, polyketone (e.g. polyetheretherketone or PEEK™), or poly-ethylene.

Fixed Length Rod Link

According to one aspect of the invention, the conducting element 80 is a non-flexible elongated member, such as a rod 85 of fixed length. Such aspect is depicted in FIG. 28. The rod 85 is attached at one end to the electromechanical actuator 20 by a joint 86, and at the other end to the vibrating surface 25, more particularly, the plate 74, by another joint 87. The joints 85, 86 accommodate small angular misalignments between the subframes 22a, 22b, and are preferably ball-and-socket joints. The rod 85 is preferably made from stainless steel, stainless steel alloy, titanium, nickel, PTFE, polypropylene, polyimide, polyketone (e.g. polyetheretherketone or PEEK™), poly-ethylene or any suitable material.

The rod 85 should fulfil the requirements for an implant such as being formed from or coated with a biocompatible and stable material, and be of suitable shape and size for insertion and placement. The rod 85 is preferably made from a rigid material, having the requisite compression and tensile properties i.e. able to resist compression and stretching in normal use. The parts of the rod 85 in contact with tissue and/or fluid may be made from any suitable biocompatible material having these properties stainless steel, stainless steel alloy, titanium, nickel, PTFE, polypropylene, polyimide, polyketone (i.e. polyetheretherketone or PEEK™), poly-ethylene or any suitable material.

Telescopic Slip Link

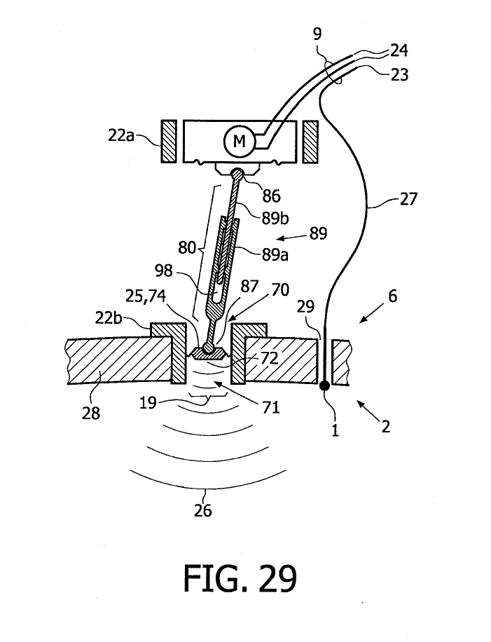

According to another aspect of the invention, the conducting element 80 is an adjustable telescopic slip link 89 whose length can be increased or decreased in a telescopic manner by the application of tensile or compression force to the ends of the link 89. Such aspect is depicted in FIG. 29. According to a preferred aspect of the invention, the adjustable slip link 89 comprises two rigid elongated members 89a, 89b each having a longitudinal axis, that are in slidable connection with each other along their longitudinal axes. The length of the slip link 89 is determined by the degree of sliding overlap of the elongated members 89a, 89b. The desired length is adjustable, but may be locked, for example, by applying a spot weld or adhesive between the respective rigid elongated members 89a, 89b. Alternatively, the length may be allowed to vary, for example, by configuring the slidable connection to expand or contract when a level of compression or tensile force applied to the ends of the slip link above a certain limit is applied; such configuration can typically be achieved with a frictional joint. The frictional joint thus allows translational movements after transplant that can absorb slow fluctuations in the sub-frame to sub-frame distance due to middle-ear pressure changes, anatomical changes (growth) etc.

In a preferred embodiment, the first rigid elongated member 89a comprises at one end, an elongated channel 98 to receive the second elongated member 89b. The channel 98 is disposed along the longitudinal axis of the first rigid elongated member 89a, and is preferably dimensioned to allow a close coupling of the second elongated member 89b. The channel 90 is of a maximum depth that allows the shortest length of the adjustable slip link 89.

The slip link 89 is attached at one end to the electromechanical actuator 20 by a joint 86, and at the other end to the vibrating surface 25, more particularly, the plate 74, by another joint 87. Said joints 86, 87 accommodate small angular misalignments between the subframes 22a, 22b, and are preferably ball joints.

The slip link 89 should fulfil the requirements for an implant such as being formed from or coated with a biocompatible and stable material, and be of suitable shape and size for insertion and placement. The slip link 89 is preferably made from a rigid material, having the requisite compression and tensile properties i.e. able to resist compression and stretching in normal use. The parts of the slip link 89 in contact with tissue and/or fluid may be made from any suitable biocompatible material having these properties stainless steel, stainless steel alloy, titanium, nickel, PTFE, polypropylene, polyimide, polyketone (e.g. polyetheretherketone or PEEK™), poly-ethylene or any suitable material.

Hinged Link

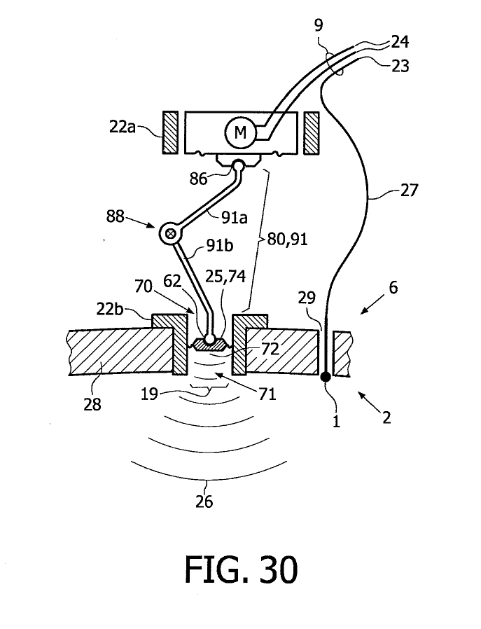

According to another aspect of the invention, the conducting element 80 is an adjustable hinged link 91 whose angle can be increased or decreased by the application of tensile or compression force to the ends of the link. Adjustment to the angle thus alters the linear distance between the ends of the link 91. Such aspect is depicted in FIG. 30. According to a preferred aspect of the invention, the adjustable hinged link 91 comprises two rigid elongated members 91a, 91b each having a longitudinal axis, that are joined to each other by a revolute joint 88. Preferably the joint 88 connects the ends or essentially the ends of each elongated member 91a, 91b. The desired linear distance between the link ends is adjustable, but may be fixed, for example, by applying a spot weld or adhesive between the respective rigid elongated members 91a, 91b, or by locking the joint 88. Alternatively, the length may be allowed to vary, for example, by configuring the revolute joint 88 to allow opening or closing of the hinge when a level of compression or tensile force applied to the ends of the link above a certain limit is applied; such configuration can typically be achieved by utilising friction in the joint. The frictional joint thus allows movements that can absorb slow fluctuations in the sub-frame to sub-frame distance due to middle-ear pressure changes, anatomical changes (growth) etc.

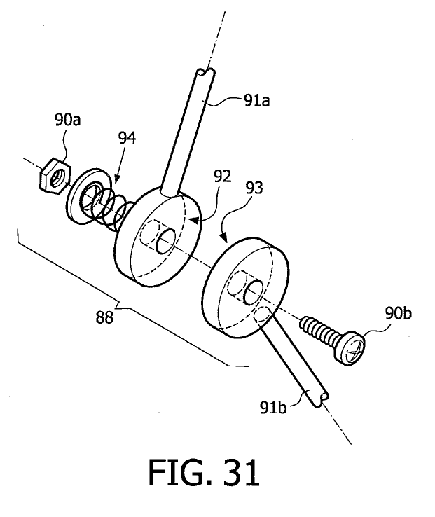

A controlled friction can be created by when the revolute joint 88 comprises two surfaces 92, 93 (FIG. 31) configured to press against each other using an adjustable force. The force can be created by a spring 94 and nut 90a and bolt 90b arrangement for example. The friction characteristics of the surfaces 92, 93 can be engineered with surface coatings (e.g. a diamond-like carbon coating) for smooth frictional slip and high wear resistance.

The hinged link 91 is attached at one end to the electromechanical actuator 20 by a joint 86, and at the other end to the vibrating surface 25, more particularly, the plate 74, by another joint 87. Said joints 86, 87 accommodate small angular misalignments between the subframes 22a, 22b, and are preferably a ball joints.

The hinged link 91 should fulfil the requirements for an implant such as being formed from or coated with a biocompatible and stable material, and be of suitable shape and size for insertion and placement. The hinged link 91 is preferably made from a rigid material, having the requisite compression and tensile properties i.e. able to resist compression and stretching in normal use. The parts of the hinged link 91 in contact with tissue and/or fluid may be made from any suitable biocompatible material having these properties stainless steel, stainless steel alloy, titanium, nickel, PTFE, polypropylene, polyimide, polyketone (e.g. polyetheretherketone or PEEK™), poly-ethylene or any suitable material.

The conducting element 80 of the above embodiments, will be of sufficient length to connect the electromechanical actuator 20 in remotely placed first sub-frame 22a with the vibrating surface 25 or output region 19 of second sub-frame 22b. The skilled person will understand that the ideal position for the placement of the first sub-frame 22a will vary from subject to subject, consequently, the length of the conducting element 80 will differ accordingly. For example, a placement of the first sub-frame 22a in the mastoid will require a shorter conducting element 80 compared with its placement in the middle ear cavity. For guidance only, the conducting element may be of a length, or may be configured to connect a distance of 3 mm, 4 mm, 5 mm, 6 mm, 7 mm, 8 mm, 9 mm, 10 mm, 11 mm, 12 mm, 13 mm, 14 mm, 15 mm, 16 mm, 17 mm, 18 mm, 19 mm, 20 mm, 21 mm, 22 mm, 23 mm, 24 mm, 25 mm, 26 mm, 27 mm, 28 mm, 29 mm, 30 mm, 31 mm, 32 mm, 33 mm, 34 mm, 35 mm, 36 mm, 37 mm, 38 mm, 39 mm, 40 mm, 41 mm, 42 mm, 43 mm, 44 mm, 45 mm, 46 mm, 47 mm, 48 mm, 49 mm, 50 mm or a value in the range between any two of the aforementioned values; preferably between 5 and 50 mm.

Vibrating Surface

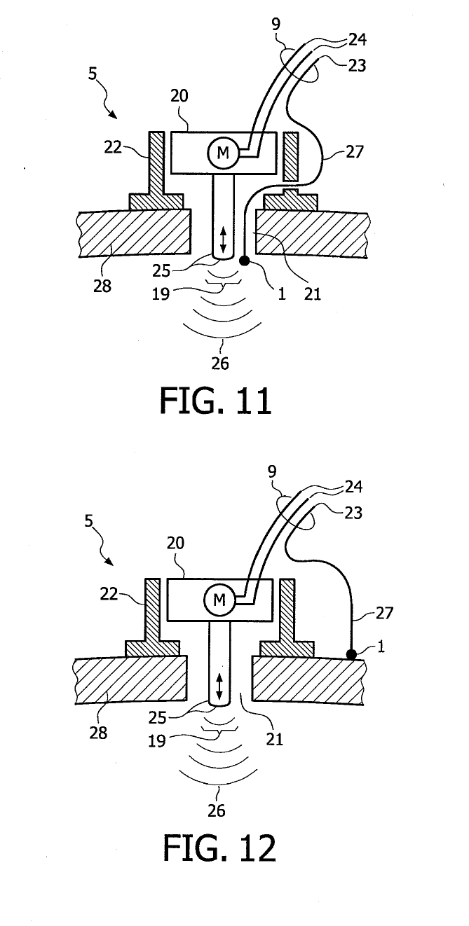

The vibrating surface 25 of the generator 5 provides vibrational energy to the output region 19. The vibrating surface 25 may be co-operatively connected (e.g. rigidly, flexibly or semi-flexibly) to the electromechanical actuator 20, or it may be extended from the electromechanical actuator 20 by a rigid, semi rigid, or fluid connection of vibration-transmitting material. The former configuration is shown, for example in FIGS. 4, 5, 6, 7, 8, 9, 10, and 26. The latter configuration is shown, for example, in FIGS. 11 to 18, where the vibrating surface 25 is extended from the electromechanical actuator 20 by means of an elongated member co-operatively connected (e.g. rigidly, flexibly or semi-flexibly) to the electromechanical actuator 20; said elongate member may be rod-like, cylindrical or any suitable shape. The latter configuration is also shown in FIGS. 21, 24, 25, 27 to 31 where the vibrating surface 25 is extended from the electromechanical actuator 20 by means of a vibrational-energy conducting element 80 that is a flexible fluid-filled tube 50 (FIG. 21, 24, 25), a flexible cable rod 53 (FIG. 27), a straight (non-flexible) rod 60 (FIG. 28), a telescopic slip link 60 (FIG. 29), or a hinged link 60 (FIG. 30). The surface 25 may have an essentially flat shape, alternatively, it may have a domed, rounded, bullet, pin or other suitable configuration.

Output Region

The output region 19 of the vibration generator 5 transmits vibrational energy to the fluid of the inner ear. The output region 19 may enter the inner ear 2. Alternatively it may contact the interface 28 between the middle ear 6 and inner ear 2 e.g. stimulate a bone in the interface 28. Alternatively it may contact the wall of the inner ear 2 e.g. stimulate a bone in the wall of the inner ear.

The output region 19 may be an aperture in the frame 22 through which vibrational energy is directed. This is shown, for example, in FIGS. 4 to 10, where a single frame encloses the electromechanical actuator 20, the vibrating surface 25 thereof is directed to the aperture. In cases where the frame comprises a first and second subframe, the first subframe houses the output region 19 through which vibrational energy is directed; said output region may be an aperture in the first sub frame 22b as shown, for example, in FIG. 26.

Alternatively, the output region 19 may be may the vibrating surface 25 of the vibration generator 5. This may be the case when the vibrating surface 25 contacts the fluid of the inner ear or contacts the interface 28 between the middle ear 6 and inner ear 2, or contact the wall of the inner ear 2. This is shown in FIGS. 11 to 18, where the vibrating surface 25 is extended from the electromechanical actuator 20 by means of a rigidly-attached elongate member. It is also depicted in FIGS. 21, 24, 25, 27, 28, 29, and 30 where the vibrating surface 25 is extended from the electromechanical actuator 20 by means of a hydraulic connection (FIGS. 21, 24, 25) or by means of a flexible cable rod 53 (FIG. 27), a straight (non-flexible) rod 60 (FIG. 28), a telescopic slip link 60 (FIG. 29), hinged link 60 (FIG. 30). According to one embodiment of the invention, the vibration generator 5 is configured so that the output region 19 can be located in a wall enclosing the inner ear, and applies vibrational stimulation to the inner ear fluid. According to one embodiment of the invention at least part of the output region 19 of the vibration generator 5 stimulates the fluid of the inner ear, preferably through a hole in the interface 28 between the middle 6 and inner ear 2. This hole may be drilled partially through the interface 28, or drilled all the way through the interface 28. The output region 19 may or may not contact the fluid of the inner ear 2. This hole may be the same hole used to attach the frame 22 or second sub-frame 22b.

According to one embodiment of the invention, at least a part of the vibrating surface 25 penetrates a lumen of the cochlea 4 (e.g. scala tympani 42, scala vestibuli 40 or the scala media 41); this is seen for example in FIGS. 11 to 18 where the vibrating surface 25 is extended by an elongate member, such that the output region 19 enters a lumen of the cochlea 4. Where a part of the vibrating surface 25 penetrates a lumen of the cochlea 4 and/or contacts the fluid of the inner ear, the vibrating surface 25 may or may not extend into the lumen. Where it does not extend, the vibrating surface 25 may be flush with the inside wall of the lumen, or recessed with the inside wall. Where it does extend, it may only extend by amount not to damage the cochlea or the intricate features inside, e.g. the fragile basilar and Reissner membranes, the spiral organ, the organ of Corti, and the sensory hair cells. According to one embodiment of the invention, the vibrating surface 25 extends into the lumen by a distance less than or equal to 1 mm, 0.8 mm, 0.6 mm, 0.4 mm, 0.2 mm, 0.1 mm, 0.08 mm, 0.06 mm, 0.04 mm, 0.02 mm, or by an amount in the range between any two of the aforementioned values. Preferably the distance is between 0.1 and 0.5 mm.

The vibration generator 5 is configured for physical attachment to a wall enclosing the inner ear 2. This means it is implantable. As such, it should fulfil the requirements for an implant such as biocompatibility, stability, and be of suitable shape and size for attachment. The parts of the vibration generator 5 in contact with tissue and/or fluid (e.g. frame 22, vibrating surface 25) may be made from any suitable biocompatible material. Where it acts as a proximal electrode 1, the conducting parts may be made from, for example, surgical steels, or platinum, iridium, titanium, gold, silver, nickel, cobalt, tantalum, molybdenum, or their biocompatible alloys. They may also be coated to lower their DC and/or AC impedance; examples of suitable coatings include porous platinum, titanium nitride with or without carbon, iridium, iridium oxide, titanium nitride with iridium oxide, or tantalum-based coatings.

The electromechanical actuator 20 may be based on any electromechanical conversion mechanism such as electromagnetic, piezoelectric, electrostatic or magnetostrictive. These mechanisms are known in the art, and some are briefly elaborated below.

An electromagnetic actuator 20 operates in a manner similar to a magnetic loudspeaker driver; the signal transmitted through lead wires 24 causes an electrical current in an actuator coil that is suspended in a magnetic field inside the actuator and mechanically coupled to an elastically suspended membrane or plate that has a vibrating surface 25 that may be in contact with the inner ear 2 fluid. The coil current in the magnetic field produces a mechanical, so-called Lorentz force on the coil, which is mechanically coupled to the elastically suspended membrane or plate and which moves the vibrating surface 25.

A piezoelectric actuator relies on the piezoelectric properties of certain crystals which, when subjected to an externally applied voltage, change shape by a small amount. Many materials like quartz, lead zirconate titanate (PZT), barium titanate, zinc oxide, and even certain polymers exhibit piezoelectricity, also called ferroelectricity, due to a charge asymmetry in the crystal structure causing a microscopic electric dipole moment. Dipoles near each other tend to be aligned in regions called Weiss domains. The domains are usually randomly oriented, but can be aligned during poling, a process by which a strong electric field is applied across the material, usually at elevated temperatures. Mechanical deformations due to piezoelectricity are typically very small, less than 0.1%. Actual applications often require additional mechanical arrangements, like bi-morphs, to amplify the deformations to more useful magnitudes. The disk bender arrangement is an example of such a mechanical amplifier well suited for the electromechanical actuator in the vibration generator 5. The disk bender comprises a thin metal plate attached along its perimeter to a generator housing and with its vibrating surface 25 exposed to the inner ear fluid. A thin piezoelectric disk is attached to the inner plate surface. One of the lead wires 24 attaches to the thin metal plate. The other lead wire attaches to a metal contact applied to the inner surface of the piezoelectric disk. An electric voltage between the metal plate and the metal contact, applied by the implanted electronic processing unit 7 through the lead wires 24, sets up an electric field in the piezoelectric disk and compresses the disk thickness. The disk bender bulges as a result, since the mechanical Poison effect in the piezoelectric material forces the disk to expand laterally, whereas the metal plate does not deform directly under the electric field. The plate bulging effect amplifies the translation distances. The deflection distance in the plate centre is typically orders of magnitude larger than the piezoelectric disk deformations.

An electrostatic actuator derives its actuation force from the electrostatic attraction between two plates at different voltages. A first plate may be formed by a thin metal plate elastically suspended along its perimeter to a generator housing and with its vibrating surface 25 exposed to the inner ear fluid. A second conductive plate is held inside the generator housing at close distance and parallel with the first plate. One of the lead wires 24 attaches to the first plate. The other lead wire attaches to the second plate. The implanted regulating unit 7 applies an electric voltage between the metal plates through the lead wires 24, which creates the electrostatic attraction force and moves the first plate with respect to the housing.

A known property of the aforementioned vibration actuators is that, besides converting electrical to mechanical energy, they may also perform the reverse operation, i.e. convert mechanical to electrical energy. That means that the vibration actuator may also be used as a microphone, for example, to sense inner-ear vibrations. Such a microphone can be part of a control loop that may automatically adjust the electrical and/or mechanical stimuli to obtain a desired vibration. This microphone feature may also enable the measurement of otoacoustic emissions directly at the cochlea producing higher fidelity measurement data compared to the current measurements in the external ear canal. Otoacoustic emissions are the acoustic response of the cochlear system to mechanical or electrical stimuli. They reflect the fundamental workings of the inner ear (Kemp D. T., “Stimulated acoustic emissions from the human auditory system,” J. Acoust. Soc. Am., vol. 64, pp. 1386-1391, 1978) and can be a powerful diagnostic and optimization tool.

Another embodiment of the invention, therefore, is a device as described herein, wherein the regulating unit 7 is configured to use readings from the electromechanical actuator 20 operating as a microphone for closed-loop control of the electrical and/or vibrational stimulation.

Certain piezoelectric, magnetostrictive and electrostatic vibration actuators, in casu the actuators that can produce a static pressure, are also sensitive to static pressure. This feature can be important in diagnostic and treatment applications. An example of such application is Ménière's Disease where the inner ear develops a slowly fluctuating static pressure that may cause fluctuating (episodic) hearing loss, vertigo, tinnitus, or aural fullness (a sense of pressure in the middle ear), for reasons that are not well understood. This static pressure can be measured with and compensated for by the vibration actuator if it is able to produce static pressures.

One embodiment of the invention, therefore, is a device as described herein, wherein the regulating unit 7 is configured to generate also a static pressure using the vibration generator 5, or more specifically the electromechanical actuator 20.

Another embodiment of the invention is a device as described herein, wherein the electromechanical actuator 20 is configured to act as a pressure sensor.

Yet another embodiment of the invention is a device as described herein, wherein the regulating unit 7 is configured to control the inner ear pressure using the vibration generator 5, or more specifically the electromechanical actuator 20.

Regulating Unit

The device may also comprise a regulating unit 7 configured to provide electrical signals for the electrodes 1, 3 and/or vibration generator 5. The regulating unit 7 may receive sound information from any type of source. These include any of the usual sources for external hearing aids, such as for example, through a wireless or wired external microphone or a Telecoil (T-coil) coupler. In one embodiment of the invention, the sound information is received through an implanted microphone. The sound information is converted by the regulating unit 7 to electrical signals for the electrodes 1, 3 and vibration generator 5. These electrical signals may be amplified. The regulating unit 7 comprises the necessary electronic components (e.g. integrated circuits, digital to analogue converts, digital signal processors, switches etc) for performing the conversion of sound information into electrical signals, which components and configurations thereof are known in the art.

The regulating unit 7 may comprise a power source either directly housed in the unit, or electrically or magnetically connected thereto. The power source may be a disposable battery, preferably a long life battery (e.g. alkaline, lithium based). The power source may be a rechargeable battery (e.g. nickel cadmium, nickel metal hydride or lithium based). The battery may be recharged by externally accessible contacts, or by an induction coil. The power source may be an induction coil; this may be coupled with an externally worn complementary coil.

It is an aspect of the invention that the regulating unit 7 may incorporate the first sub-frame 22a of the vibration generator 5, as shown, for example, in FIG. 23. According to the illustrated embodiment, a tube 80 carries a hydraulic connection to the output region of the second sub-frame 22b.

The regulating unit 7 is preferably implantable. As such, it should fulfil the requirements for an implant such as biocompatible and stable housing, and be of suitable shape and size for insertion and placement. The parts of the regulating unit 7 in contact with tissue and/or fluid may be made from any suitable biocompatible material. Where it acts as a distal electrode 3, the conducting parts may be made from, for example, surgical steels, or platinum, iridium, titanium, gold, silver, nickel, cobalt, tantalum, molybdenum, or their biocompatible alloys. They may also be coated to lower their DC and/or AC impedance; examples of suitable coatings include porous platinum, titanium nitride with or without carbon, iridium, iridium oxide, titanium nitride with iridium oxide, or tantalum-based coatings.

The regulating unit 7 may be configured to perform some sound processing tasks. In one embodiment of the invention, the regulating unit 7 processes received sound information and translates it into electrical signals carried by the proximal 1 and distal 3 electrodes, which are able to trigger nerves to fire neural signals (i.e. action potentials). Although the electrical signals are derived from sound, they do not resemble audio signals. Electrical signals may be, but not limited to, bursts of short bi-phasic pulses i.e. positive current pulse followed by an equal charge negative pulse. Typically, these pulses have a higher amplitude when the sound information is louder. They are typically 10-100 μs long with ps edge transients, i.e. much shorter than audio signals. Such signals and processing thereto is known in the art, and the present method encompasses any processing tasks which convert sound information into signals suitable for stimulation of the auditory nerve.

According to one aspect of the invention, the regulating unit 7 is configured to translate sound information into electrical signals able to trigger nerves to fire neural signals, which electrical signals are provided to the electrodes 1, 3. According to another aspect of the invention, the regulating unit 7 is configured to translate full audio frequency spectrum into said electrical signals. According to one aspect of the invention, the regulating unit 7 is configured to enhance or suppress one or more bands of frequency within said full audio frequency (multi-band filtering), prior to translation.

In one embodiment of the invention, the regulating unit 7 processes received sound information and converts it into signals for sending to the vibration generator 5 which in turn produces the corresponding mechanical vibrations in the inner ear fluid. The signal may be amplified. Such signals may represent full audio spectrum sound. Alternatively, the regulating unit 7 processes may provide only sound in a narrow spectrum e.g. provide only higher (e.g. higher than 2500 Hz) frequency or lower (e.g. less than 2500 Hz) frequency sound to the vibration generator 5, which frequency ranges are exemplified below.

According to one aspect of the invention, the regulating unit 7 processes received sound information for the vibration generator using a multi-band filtering and processing; this many mean the vibration generator will receive full audio spectrum whereby certain frequency band frequencies are be enhanced or suppressed e.g. a limited number of high frequency bands enhanced.

According to one aspect of the invention, the regulating unit 7 is configured to provide full audio frequency spectrum to the vibration generator 5. According to another aspect of the invention, the regulating unit 7 is configured to enhance or suppress one or more bands of frequency within said audio frequency spectrum (multi-band filtering).

In one embodiment of the invention, the regulating unit processes received sound information by splitting it into two frequency bands—one comprising higher frequency signals and one comprising lower frequency signals. The crossover frequency may be between 500 Hz and 5 kHz depending on the patient's condition. The higher frequency signals may be equal to or greater than 500 Hz, 600 Hz, 700 Hz, 800 Hz, 900 Hz, 1 kHz, 2 kHz, 3 kHz, 4 kHz, 5 kHz, 6 kHz, 7 kHz, 8 kHz, 9 kHz, 10 kHz, 11 kHz, 12 kHz, 13 kHz, 14 kHz, 15 kHz, 16 kHz, 17 kHz, 18 kHz, 19 kHz, 20 kHz, or a value in the range between any two of the aforementioned values. Preferably the higher frequency signals are between 2 kHz and 14 kHz. The lower frequency signals may be equal to or less than 500 Hz, 400 Hz, 300 Hz, 200 Hz, 100 Hz, 80 Hz, 60 Hz, 40 Hz, 20 Hz, 10 Hz. Preferably the lower frequency signals are between 100 Hz and 500 kHz. The low-frequency sound information may be processed by the regulating unit 7, and provided as a signal to the vibration generator 5 which in turn produces the corresponding mechanical vibrations in the inner ear fluid. The high-frequency sound information may be processed by the regulating unit 7, and provided as electrical signals for triggering neuronal signalling to the electrodes 1, 3 for electrical stimulation of the cochlea. The high-frequency sound information may be processed according to techniques known in the art as mentioned already. This may involve signal rectification, amplitude envelope detection, compression and translation (i.e. translation of the band-filtered and compressed audio into bursts of microsecond pulses) to create electrical stimulation.

As mentioned above, the extent of mechanical and electrical stimulation will depend on the condition of the subject. Some will benefit from simultaneous mechanical and electrical stimulation, others may only need mechanical stimulation, and others only electrical stimulation. Some patients will benefit from full-audio vibration stimulation, other will require enhancement of certain frequencies. Some patients may need complex multi-band audio processing. The precise requirement of each subject may be adjusted and maintained by the regulating unit.

According to one aspect of the invention, the regulating unit 7 is programmable so that the sound-processing configuration (e.g. split between mechanical and electrical stimulation, processing algorithms are used in the mechanical and the electrical signal path, threshold levels, gain settings, filter parameters, compression parameters, electrode selection etc) can be changed depending on how the unit is programmed. The programming can be prepared to suit the patient's condition. The unit 7 may comprise a memory storage device for storing such programmable configurations. The regulating unit 7 comprises the necessary electronic components (e.g. integrated circuits, memory chips, etc) for performing programmability, which components and configurations thereof are known in the art.

The programmable configuration may be entered into the regulating unit 7 via a wireless link. This wireless link can be, for example, an inductive-powering link by means of field modulation or backscattering, a dedicated radio link, a dedicated induction link separate from the powering link, or an infrared link. The regulating unit 7 comprises the necessary electronic components (e.g. integrated circuits, digital signal processors, antennas, etc) for performing the conversion of sound information into signals, which components and configurations thereof are known in the art.

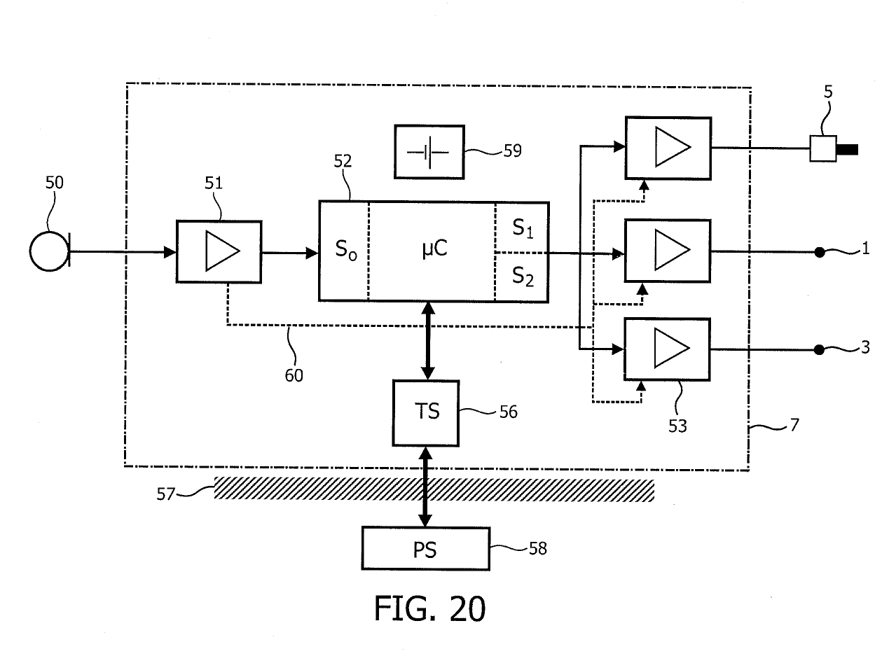

The processing tasks, wireless capability and optional programmability functions are performed using an arrangement of components disposed within the regulating unit 7. FIG. 20 shows a possible configuration of components within the regulating unit 7. Sound is picked up via one or more microphones 50 and is converted into electrical signals. The analogue electrical sensor signals are routed to modules 51 in which they are preprocessed, especially preamplified, and converted into digital signals (ND). This preprocessing can be provided by, for example, analogue linear or nonlinear pre-amplification and filtering (for example, anti-aliasing filtration).

The digitised sound information is further processed in a microcontroller 52 (pC). The microcontroller 52 contains a read-only-memory area S0 which cannot be overwritten, in which the instructions and parameters necessary for “minimum operation” of the system are stored, and storage areas S1 and S2 in which the operating software of the intended function or functions of the regulating unit 7 are stored. The rewriteable program storages S1 and S2 for storing the operating software can be based on EEPROM or on static RAM cells, and in the latter case, provisions may be made within the regulating unit for this RAM area to always be powered.

The digital output signals of the microcontroller 52 are converted using digital-analog converters (D/A) 53 into analogue signals and amplified and then supplied to the stimulating electrodes 1, 3 and the vibration generator 5.

The microcontroller 52 executes the intended function of the hearing implant. This includes audio signal processing described above and optionally also signal generation in the case of a system with additional tinnitus masker or noiser function. Furthermore, the microcontroller 52 may contain software modules which provide for dual control of the stimulating electrodes 1, 3 and the vibration generator 5 in such a manner that the spectral, time, amplitude- and phase-referenced transducer or stimulating electrode signal properties are configured such that optimum hearing success is achieved for the pertinent patient. These software modules can be designed to be static and dynamic. A static design is intended to mean that the software modules, based on scientific findings, are stored once in the program storage of the microcontroller 52 and remain unchanged. Dynamic means that these software modules are “able to learn”, in order to approach as optimally as possible the desired hearing result in a time iterative manner. This means that the software modules can be designed to be adaptive, and parameter matching is done by training by the implant wearer and optionally using other aids such as rehabilitation programs. Furthermore, a software module can be provided which approximates hearing supply as optimum as possible based on an adaptive neural network. Training of this neural network can take place again by the implant wearer and/or using other external aids.

According to one aspect of the invention, the microcontroller 52 communicates via a bidirectional data bus 55 and a telemetry system (TS) 56 wirelessly (for example, via inductive coupling) through the closed skin indicated at 57 with an external programming system (PS) 58. The programming system 58 can be a PC-based system with corresponding programming, processing, display and administration software. Via this telemetry interface, the operating software of the regulating unit 7 which is to be changed or completely replaced is transmitted. Thus, for example, simple verification of software transmission can be done by a reading process via the telemetry interface before the operating software or the corresponding signal processing portions of this software are transmitted into the program storage areas S1 and S2 of the microcontroller 52 via a data bus 55. Furthermore, the working program for the microcontroller 52 can be changed or replaced in whole or in part via the telemetry interface using the external unit 58.

According to another aspect of the invention, the microcontroller 52 controls within the regulating unit 7, via the bidirectional data bus 60, the ND converters 51 of the sensor preprocessing, the D/A converters 53 for control of the stimulating electrodes 1, 3 and the vibration generator 5. The D/A converters 53 can also be partially or entirely omitted when there are digitally controlled power sources for the stimulating electrodes and/or, in case a vibration generator 5 is used, for example, a pulse width-modulated serial digital output signal of the microcontroller 52 is transmitted directly to the vibration generator 5. Via the data bus 60, program parts or entire software modules can also be transferred between an external unit and the microcontroller 52.

The regulating unit 7 may also comprise a primary or secondary battery cell 59 that supplies the individual components with electrical operating energy.

According to one embodiment if the invention, the regulating unit 7 may have a measurement amplifier which can read electrode voltages (distal and proximal) which can be used by the implant in a feedback loop to automatically adjust the stimulation signals:
Voltages on the stimulating electrodes during electrical stimulation allow assessing electrode impedance;
Voltages on the non-stimulating electrodes during and right after electrical stimulation allow measuring the electrical response of the neural system;
Voltages on the non-stimulating electrodes during and right after vibrational stimulation allow measuring the electrical response of the neural system;

Other Components

The device may also comprise other components as would be understood by the person skilled in the art. For example, it may comprise electrical leads 8, 9, 10, 23, 24 that connect the electrodes 1, 3 and vibration generator 5 to a regulating unit 7. Connectors may be included on the electrodes 1, 3, vibration generator 5 and/or regulating unit 7 to allow the replacement of these components while leaving the electrical leads 8, 9, 10, 23, 24 in situ. Connectors may be included in the leads 8, 9, 10, 23, 24 to allow easier replacement of the electrodes 1, 3, vibration generator 5 and/or regulating unit 7 while leaving sections of the electrical leads 8, 9, 10, 23, 24 in situ.

The device may take advantage of wireless connectivity, for example, to pass information between the microphone and the regulating unit 7. Alternatively, or in addition, the device may also use wireless connectivity to transfer data between the regulating unit 7 and an external device. The external device may be capable of programming the regulating unit 7, receiving data from the regulating unit, or controlling the regulating unit.

The wireless link can be, for example, an inductive-powering link by means of field modulation or backscattering, a dedicated radio link, a dedicated induction link separate from the powering link, an infrared link or any wireless link known in the art. It can adopt a technical standard for data transfer such as Wi-fi, ZigBee or Bluetooth.

Configurations

The electrodes, vibration generator, and regulating means, described above, can be implement in a variety of configurations, which are within the knowledge of the skilled artisan. Variations include the configurations of the proximal electrode 1 and vibration generator 5 which are elaborated below.

In FIG. 5, the proximal electrode 1 is disposed in the same opening as the vibration generator 5. In FIG. 6, the proximal electrode 1 is disposed in an opening 29 adjacent to the vibration generator 5, whereby the opening passes all the way through the interface 28. In FIG. 7, the proximal electrode 1 is disposed in an opening 29 adjacent to the vibration generator 5, whereby the opening passes partially through the interface 28. In FIG. 8, the proximal electrode 1 is disposed on the surface 28 of the wall, and adjacent to the vibration generator 5. In FIG. 9, the proximal electrode 1 is disposed within the frame 22 of the vibration generator 5. In FIG. 10, the proximal electrode 1 is disposed on the vibrating surface 25 of vibration generator 5.

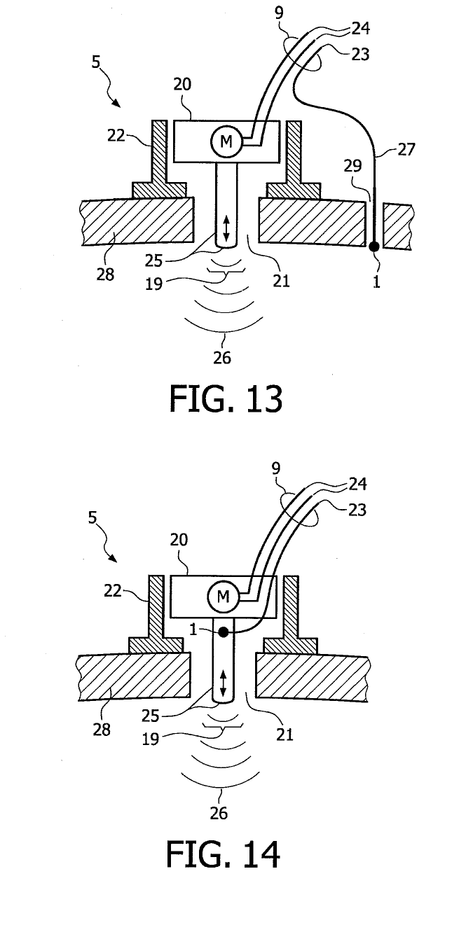

In FIGS. 11, 12 and 13, the vibrating surface 25 of the vibration generator 5 is connected to the electromechanical actuator 20 by means of a rigidly attached elongate member; at least part of the output region 19 extends through the small hole 21 of the interface 28. The longitudinal axis of the elongated member is linear. The elongated member may have a cylindrical or a polygonal (e.g. 3, 4, 5, 6, 7, 8 or more sided) surface. The elongated member may act on an exposed lining, for example the endosteal lining of the inner ear fluid spaces or directly on the inner ear fluid in order to transfer the vibration energy. The proximal electrode 1 may be implanted in the said small hole 21 (FIG. 11). It may be implanted in a second small hole 29 in an inner ear part. This small hole may be artificially drilled through a bony wall, or it may be an oval window (FIG. 13); the hole may pass all the way through the interface 28 or pass partially through the interface 28. It may be implanted on the surface of either a bony wall, or oval window (FIG. 12).

The frame 22 is fixed to the solid tissue (e.g. bone) surrounding the said hole 21, and holds the vibration generator 5 and therefore the output region 19 in place and aligned to the small hole 21.

In FIG. 14, the vibrating surface 25 of the vibration generator 5 is extended by an elongated member that passes through the small hole 21; the elongated member and vibrating surface 25 are made out of an electrically conductive material and also function as a proximal electrode. The frame 22 is again fixed to the solid tissue (e.g. bone) surrounding the said hole 21, and holds the vibration generator 5 and output region 19 in place and aligned to the small hole 21.

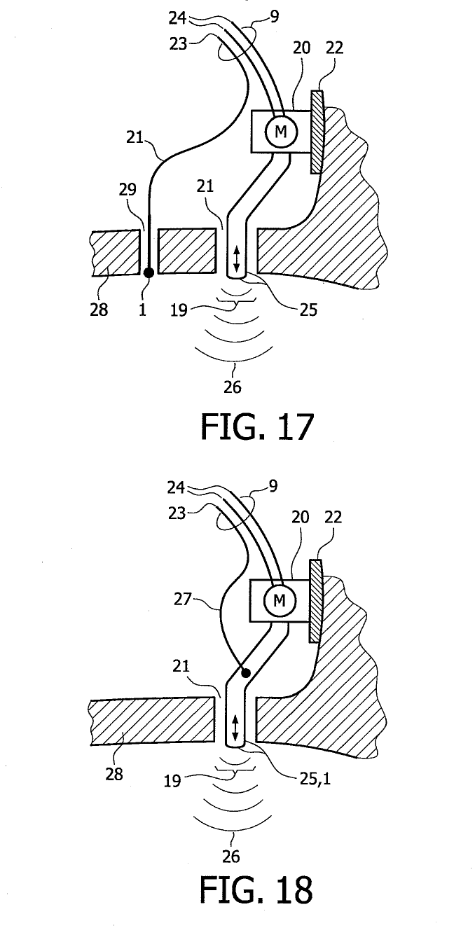

In FIGS. 15, 16, 17 and 18 the vibrating surface 25 is extended by an elongated member, which longitudinal axis is not linear. In this instance, the longitudinal axis is shaped to allow attachment of the frame 22 of the vibration generator 5 to a structure that is not the interface 28. The shape of the non-linear elongated member can be any, for example, the longitudinal axis may be curved, angled, or have several angled joins or curves. In FIGS. 15, 16 and 17, the frame 22 is fixed to solid tissue (e.g. bone) at some distance from the said hole 21, and holds the vibration generator 5 in place and aligned with the small hole 21. The proximal electrode 1 may be implanted in the said small hole 21 (FIG. 15). It may be implanted in a second small hole 29 in an inner ear part. The small hole may be artificially drilled in the bony wall, or it may be an oval window (FIG. 17). The artificially drilled hole may pass all the way through the interface 28 or pass partially through the interface 28. The proximal electrode 1 may be implanted on the surface of either a bony wall or an oval window (FIG. 16). In FIG. 18, the elongated member and vibrating surface 25 are made out of an electrically conductive material to also function as the proximal electrode 1. Therefore, a separate attachment of the proximal electrode 1 is not necessary in this embodiment.

In FIGS. 21 to 30, the frame comprises a first sub-frame 22a that supports the electromechanical actuator 20 and a second sub-frame 22b configured for attachment at the interface between the middle 6 and inner ear 2, or between the mastoid and the inner ear 2, and which provides the output region 19, wherein the vibration energy from the electromechanical actuator 20 is directed to the output region 19 via a vibrational-energy conducting element 80. The second sub-frame 22b forms a passage 72 having a receiving end 70 to receive vibrational energy from the conducting element 80, and a transmitting end 71 where vibrational energy is directed towards the inner ear fluid,

In FIGS. 21, 22, 23, 24, 25 and 26, the conducting element 80 is depicted as a flexible tube 84 containing a non-compressible liquid or gel 81. In FIG. 26 the vibrating surface 25 is disposed in the first sub-frame. In FIGS. 21, 24, 25, 27, 28, 29, and 30, the second sub-frame 22b is disposed with the vibrating surface 25 in the passage 72, optionally in connection with a region towards or at the transmitting end 71. In FIG. 21 the vibrating surface 25 is a flexible or flexibly suspended membrane 73 in sealing connection with the transmitting end 71 of the passage 72, and in hydraulic connection with the electromechanical actuator 20. In FIG. 24 the vibrating surface 25 is formed from a sliding piston 75 in hydraulic connection with the electromechanical actuator 20. In FIG. 25 the vibrating surface 25 comprises a flexibly suspended rigid membrane 105 in sealing connection with the transmitting end 71 of the passage 72, and in hydraulic connection with the electromechanical actuator 20, and a pin 101 attached to said which protrudes from the transmitting end 71 of the passage 72.

In FIGS. 27, 28, 29, and 30, the conducting element 80 is a mechanical link. In FIG. 27, the conducting element 80 is a cable link, comprising a flexible cable 83 housed in an essentially stationary sleeve 82, which cable 83 is configured to move within the sleeve 82, while maintaining a coaxial relation therewith. The vibrating surface 25 is formed from a sliding piston 75 in mechanical connection with the electromechanical actuator 20. In FIG. 28, the conducting element 80 is a non-flexible, elongated rod 85. The vibrating surface 25 is a flexibly suspended plate 74 in mechanical connection with the electromechanical actuator 20. In FIG. 29, the conducting element 80 is an adjustable telescopic slip link 89. The vibrating surface 25 is a flexibly suspended plate 74 in mechanical connection with the electromechanical actuator 20. In FIG. 30, the conducting element 80 is an adjustable hinged link 91. The vibrating surface 25 is a flexibly suspended plate 74 in mechanical connection with the electromechanical actuator 20.

When electrical stimulation is applied across the distal 3 and proximal electrodes 1, the inner ear neural structures are stimulated. When electrical stimulation is combined with vibrational stimulation, there is a significant improvement in hearing experienced by a subject. Unlike with conventional pure electrical cochlea stimulation, or with hybrid stimulation using elongated electrodes inserted in the cochlea, the improvement produced by the present invention is complemented by no or reduced loss in residual hearing. This can be a significant advantage to certain otoacoustical pathologies.

The invention also allows the specialist (e.g. surgeon) to implant an electrical and a mechanical stimulatory hearing aid in a single procedure, when he does not have the foreknowledge of which stimulation would be the most effective. After the surgery, parameters such as the balance between mechanical and electrical stimulation, the signal processing algorithms and settings, can be carefully tuned to the pathology of the specific patient, and retuned periodically over the lifetime of the implant in cases with progressing hearing loss. For example, in case of locally damaged inner ear structures, mechanical stimulation can be greatly impaired. In patients with presbyacousis where the sensory cells (hair cells) for sensing the high frequencies are damaged, the underlying neural structures may still be functional and can be electrically stimulated to transfer high frequency acoustical information. Thus, the invention would provide both electrical and vibrational stimulation, these would be tested by the specialist (e.g. audiologist), and the proportions of electrical and vibrational stimulation adjusted according to the extent of the damage.

Kit

One embodiment of the present invention is a kit comprising one or more of the following components:
at least one (e.g. 1, 2, 3, 4 or 5) proximal electrode 1,
at least one (e.g. 1, 2, 3, 4 or 5) distal electrode 3,
at least one (e.g. 1, 2, 3, 4 or 5) vibration generator 5,
one or more electrical leads 8, 9, 10, 23, 24, which may or may not be disposed with a connector for electrical leads,
a regulating unit 7, and
one or more surgical tools.

As mentioned elsewhere, the proximal electrode and vibration generator may be comprised in a single unit.

The kit may also comprise surgical tools and instructions for use.

The kit may provide components specific to a particular size of implant. Alternatively, it may provide a range of different sizes, to accommodate different attachment sites.

Method

The present invention also relates to a method for improving hearing of a subject, by:
electrically stimulating the cochlea using two or more electrodes none of which pass along the scala tympani 42, scala vestibuli 40 or the scala media 41, in combination with
mechanically stimulating the fluid of the inner ear.

One embodiment of the present invention is a method for improving hearing in a subject comprising:
implanting a vibration generator (5) comprising an output region (19), such that said output region is located in a wall enclosing the inner ear, and applies vibrational stimulation to the inner ear fluid,
implanting in a wall enclosing the inner ear 2, a proximal electrode 1, proximal to the output region 19 of the vibration generator 5, and
implanting a distal electrode 3 such that it makes electrical contact with the auditory nerve 32.

The description above in respect of the device applies also to the present method embodiments, and is elaborated below.

The properties of the proximal electrode 1 are described above. Preferably, the proximal electrode 1 is attached to the outside of the wall enclosing the inner ear, i.e. on the non-fluid-filled side of the wall. Preferably, the proximal electrode is attached at the interface between the middle 6 and inner ear 2; the interface may include the promontorium. Preferably, it is attached at the interface between the middle 6 and inner ear 2, where there is a bony part. Preferably, the proximal electrode 1 is attached at the interface between the middle 6 and inner ear 2, the bony wall accessing the scala vestibuli 40 or the scala timpani 42. Preferably, the proximal electrode 1 is attached to an artificially drilled hole in the bony wall accessing the scala vestibuli 40 (FIG. 1) or to the oval window 12 (FIG. 2). The proximal electrode 1 may attach either to the surface of the wall, to a small hole drilled partially through the wall, or to a small hole drilled all the way through the wall.

According to one embodiment of the invention, the proximal electrode 1 penetrates a lumen of the cochlea 4 (e.g. the scala tympani 42, scala vestibuli 40 or the scala media 41) and contacts the fluid of the lumen. Where the electrode is pin-shaped, a longitudinal axis of the electrode may be divergent from a longitudinal centreline of a cochlea 4 lumen. In other words, a pin-shaped electrode may not lie along the passage of a lumen of the cochlea 4. The longitudinal axis and centreline may preferably be about perpendicular. This configuration is distinct from the prior art (e.g. FIG. 3) as previously explained.

Where the proximal electrode 1 penetrates the lumen of the cochlea 4 and contacts the fluid of the lumen, the electrode may or may not extend into the lumen. Where it does not, the electrode may be flush with the inside wall of the lumen, or recessed with the inside wall. Where it does, it may only extend by amount not to damage the fragile basilar and Reissner membranes, the spiral organ, the organ of Corti, or the sensory cells (hair cells) inside the cochlea. According to one embodiment of the invention, the proximal electrode 1 extends into the lumen by a distance less than or equal to 2 mm, 1.8 mm, 1.6 mm, 1.4 mm, 1.2 mm, 1 mm, 0.8 mm, 0.6 mm, 0.4 mm, 0.2 mm, 0.1 mm, 0.08 mm, 0.06 mm, 0.04 mm, 0.02 mm, or by an amount in the range between any two of the aforementioned values. Preferably the distance is between 0.1 and 0.5 mm.

The number of proximal electrodes attached may be 1, 2, 3, 4, 5, 6, 7, 8, 9, 10 or more. The number of proximal electrodes may equal the number of distal electrodes.

According to one aspect of the invention, a proximal electrode 1 is attached to a wall enclosing the inner ear 2, in close proximity to the output region 19 of the vibration generator 5. This configuration means the output region 19 of the vibration generator 5 and proximal electrode 1 are close together, so making implantation easier. The proximal electrode 1 may be attached to the surface of the wall, adjacent to the output region 19 of the vibration generator 5; this embodiment is seen, for example, in FIGS. 8, 12 and 16. The vibration generator 5 (frame 22, or subframe 22b) and proximal electrode 1 may share the same hole; this embodiment is seen, for example, in FIGS. 5, 11 and 15. The proximal electrode 1 may be attached to the wall, adjacent to the output region 19 of the vibration generator 5, and contact the inner ear fluid; this embodiment is seen, for example, in FIGS. 6, 13, 17, 21, and 24 to 30 where the proximal electrode 1 is disposed in a separate small hole 29. The proximal electrode 1 may be attached to the wall, adjacent to the output region 25 of the vibration generator 5, in a small hole partially drilled through a wall enclosing the middle ear; this embodiment is seen, for example, in FIG. 7 where the proximal electrode 1 is disposed in a second small hole 21 partially drilled through the interface 28. Alternatively, the proximal electrode 1 may be comprised in the vibration generator 5; this embodiment is seen, for example, in FIG. 9 (as part of the frame 22) or FIGS. 10, 14 and 18 (as part of the output region 25). According to one aspect of the invention, the output region 19 of the vibration generator 5 and the proximal electrode 1 are attached so as to be less than or equal to 10 mm, 9.5 mm, 9.0 mm, 8.5 mm, 8.0 mm, 7.5 mm, 7.0 mm, 6.5 mm, 6.0 mm, 5.5 mm, 5.0 mm, 4.5 mm, 4.0 mm, 3.5 mm, 3.0 mm, 2.5 mm, 2.0 mm, 1.0 mm, 0.1 mm, 0.01 mm apart, or a distance apart that is in the range between any two of the aforementioned values. Preferably the distance is between 0.01 and 5 mm.

The properties of the distal electrode are described above. The distal electrode 3 is placed apart from the proximal electrode 1, and is implanted to make electrical contact with the auditory nerve 32. It may or may not be in physical contact with the auditory nerve 32 to achieve this. Where it is in physical contact with the auditory nerve 32, it may be attached thereto.

Where it is not in physical contact with the auditory nerve 32, it may be attached to a wall enclosing the cochlea 4. In which case, the distal electrode 3 is preferably configured for attachment to the outside of the wall enclosing the cochlea 4, i.e. on the non-fluid-filled side of the wall. The distal electrode 3 may attach either to the surface of the wall, to a small hole drilled partially through the wall, or through a small hole drilled all the way through the wall.

According to one embodiment of the invention, the distal electrode 3 is attached to the cochlea 4 so that it penetrates a lumen of the cochlea 4 (e.g. scala tympani 42, scala vestibuli 40 or the scala media 41) and contacts the fluid of the lumen. In this embodiment the longitudinal axis of the implanted electrode may be divergent from a longitudinal centreline of a cochlea 4 lumen. This is distinct from the prior art (e.g. FIG. 3) as described above.

Where the distal electrode 3 penetrates a lumen of the cochlea 4 and contacts the fluid of the inner ear, the electrode may or may not extend into the lumen. Where it does not, the electrode may be flush with the inside wall of the lumen, or recessed with the inside wall. Where it does, it may only extend by amount not to damage the fragile basilar and Reissner membranes, the spiral organ, the organ of Corti, or the sensory cells (hair cells) inside the cochlea. According to one embodiment of the invention, the distal electrode 3 extends into the lumen by a distance less than or equal to 2 mm, 1.8 mm, 1.6 mm, 1.4 mm, 1.2 mm, 1 mm, 0.8 mm, 0.6 mm, 0.4 mm, 0.2 mm, 0.1 mm, 0.08 mm, 0.06 mm, 0.04 mm, 0.02 mm, or by an amount in the range between any two of the aforementioned values. Preferably the distance is between 0.1 and 1.0 mm.

Where the distal electrode 3 is not in physical contact with the cochlea 4, it is implanted so as to retain electrical contact with the auditory nerve or the neural elements inside the cochlea 4. This may mean the cochlea 4 can be electrically stimulated by said distal electrode 3. This may also mean that the distal electrode 3 is implanted so that electrical impedance between the distal electrode 3 and the inner ear fluid 4 at 1 kHz is less than or equal to 100 000 ohms, 80 000 ohms, 60 000 ohms, 40 000 ohms, 20 000 ohms, 10 000 ohms, 8 000 ohms, 5 000 ohms, 2 000 ohms, 1 000 ohms, 800 ohms, 600 ohms, 400 ohms, 200 ohms, 100 ohms, 50 ohms, or a value in the range between any two of the aforementioned values. Preferably the impedance is between 10 and 10 000 ohms.

According to one aspect of the invention, the distal 3 and/or proximal 1 electrodes are implanted so that the electrical impedance between the distal electrode 3 and proximal electrode 1 at 1 kHz is less than or equal to 100 000 ohms, 80 000 ohms, 60 000 ohms, 40 000 ohms, 20 000 ohms, 10 000 ohms, 8 000 ohms, 5 000 ohms, 2 000 ohms, 1 000 ohms, 800 ohms, 600 ohms, 400 ohms, 200 ohms, 100 ohms, 50 ohms, or a value in the range between any two of the aforementioned values. Preferably the impedance is between 10 and 10 000 ohms.

According to one aspect of the invention, the distal 3 and/or proximal 1 electrodes are implanted so that the electrical resistance between the distal electrode 3 and the proximal electrode 1 is less than or equal to 100 000 ohms, 80 000 ohms, 60 000 ohms, 40 000 ohms, 20 000 ohms, 10 000 ohms, 8 000 ohms, 5 000 ohms, 2 000 ohms, 1 000 ohms, 800 ohms, 600 ohms, 400 ohms, 200 ohms, 100 ohms, 50 ohms, or a value in the range between any two of the aforementioned values. Preferably the resistance is between 10 and 10 000 ohms.

According to one embodiment of the invention, the distal electrode 3 is attached in the vicinity of the inner ear 2. As mentioned above, it may be in contact with the cochlea 4, on the non-fluid-filled side of the wall. It may make contact with the auditory nerve. For instance, it may be implanted in a hole accessing the singular nerve (posterior ampullary nerve) canal that passes vestibular nerve fibres to the auditory brain stem, providing a low-impedance connection to the auditory nerve. Alternatively, the distal electrode 3 may be remote from the cochlea 4. According to one aspect of the invention, the distal electrode 3 may be disposed within an implanted regulating unit 7 as described above. For example, it may be disposed as an electrically conductive patch on the exterior housing of the regulating unit 7. Alternatively, the distal electrode may be the casing itself of the regulating unit 7.

The number of distal electrodes attached may be 1, 2, 3, 4, 5, 6, 7, 8, 9, 10 or more. The number of distal electrodes may equal the number of proximal electrodes.

The vibration generator 5 is implanted such that its' output region is located in a wall enclosing the inner ear, and can apply vibrational stimulation to the inner ear fluid. The frame 22, or second subframe 22b where present, of the vibration generator 5 is generally attached to a wall enclosing the middle ear 6. The wall is usually solid tissue (e.g. bone). Preferably, the frame 22, more particularly, the second subframe 22b is attached to the outside of the wall enclosing the inner ear 2, i.e. on the non-fluid-filled side of the wall. Preferably, the frame 22, more particularly, the second subframe 22b of the vibration generator is attached at the interface between the middle 6 and inner ear 2. Preferably, the frame 22, more particularly, the second subframe 22b of the vibration generator 5 is attached at the interface between the middle 6 and inner ear 2, where there is a bony part. Preferably, the frame 22, more particularly, the second subframe 22b is attached at the interface between the middle 6 and inner ear 2, on the bony wall accessing the scala vestibuli 40 or the scala tympani 42. Preferably, the frame 22, more particularly, the second subframe 22b of the vibration generator 5 is attached to an artificially drilled hole in the bony wall accessing the scala vestibuli (FIG. 1) or to the oval window 12 (FIG. 2). The frame 22, more particularly, the second subframe 22b may attach either to the surface of the wall, to a small hole drilled partially through the wall, or to a small hole drilled all the way through the wall. According to yet another embodiment of the invention, the frame 22 more particularly, the second subframe 22b is attached at the interface between the inner ear 2 and the mastoid region. According to yet another embodiment of the invention, the frame 22 more particularly, the second subframe 22b is attached at the interface between the inner ear 2 and the mastoid region where there is a bony part.

According to one embodiment of the invention, the frame 22, more particularly, the first subframe 22a is attached to a wall enclosing the middle ear 6, which wall is not an interface 28 between the middle 6 and inner ear 2. This is exemplified in FIGS. 15 to 18, where the wall is adjacent to said interface 28.

According to one embodiment of the invention, the frame 22 more particularly, the first subframe 22a, is embedded in a cavity machined in a bony wall enclosing the middle ear 6, which wall is not an interface 28 between the middle 6 and inner ear 2, e.g. in the mastoid bone.

According to another aspect of the invention, the frame 22 more particularly, the first subframe 22a, is embedded in a cavity 100 as shown, for example, in FIG. 22 where it is implanted in the mastoid. As already mentioned, we have found that the inner-ear vestibule can be accessed surgically from behind the ear via the mastoid, so allowing convenient implantation. According to another yet another aspect of the invention, the first sub-frame 22a of the vibration generator 5 is incorporated within the housing of the regulating unit 7, as shown, for example, in FIG. 23.

According to one embodiment of the invention, vibration generator 5 is attached such that at least a part of the vibrating surface 25 penetrates a lumen of the cochlea 4 (e.g. scala tympani 42, scala vestibuli 40 or the scala media 41) and contacts the fluid of the lumen); this is seen for example in FIGS. 11 to 18 where the vibrating surface 25 is extended by an elongated member, such that the output region 19 enters a lumen of the cochlea 4. Where a part of the vibrating surface 25 penetrates a lumen of the cochlea 4 and contacts the fluid of the lumen, the vibrating surface 25 may or may not extend into the lumen. Where it does not extend, the vibrating surface 25 may be flush with the inside wall of the lumen, or recessed with the inside wall. Where it does extend, it may only extend by amount not to damage the cochlea or the intricate features inside, e.g. the fragile basilar and Reissner membranes, the spiral organ, the organ of Corti, and the sensory hair cells. According to one embodiment of the invention, the vibrating surface 25 extends into the lumen by a distance less than or equal to 1 mm, 0.8 mm, 0.6 mm, 0.4 mm, 0.2 mm, 0.1 mm, 0.08 mm, 0.06 mm, 0.04 mm, 0.02 mm, or by an amount in the range between any two of the aforementioned values. Preferably the distance is between 0.1 and 0.5 mm.

The present invention may further comprise the step of implanting a regulating unit, and connecting said electrodes and vibration generator to said unit using one or more wire cables. The properties of a regulating unit are described above, one or more of which may be implemented into the present method.

The method of the present invention includes the steps which lead to implantation of the configurations depicted in FIGS. 1 to 31 and which are elaborated elsewhere herein.

It will be within the competence of the skilled person to carry out the steps of method or construct the above described device. Those skilled in the art will recognise, or be able to ascertain using no more than routine substitutions, many equivalents to the specific embodiments of the invention described herein.

1. An implantable device for improving hearing in a subject comprising:
a vibration generator comprising an output region configured to apply vibrational stimulation to the inner ear fluid of the subject,
a proximal electrode configured for physical attachment to a wall enclosing the inner ear at a location proximal to the output region of the vibration generator, and
a separate distal electrode configured to make electrical contact with the auditory nerve of the subject.

2. Device according to claim 1, wherein the vibration generator comprises:
an electromechanical actuator,
a vibrating surface co-operatively connected to the electromechanical actuator, wherein the vibrating surface provides vibrational energy, and
a frame configured to position the vibrating surface to direct vibrational energy therefrom to the output region.

3. Device according to claim 2, wherein the frame is configured for physical attachment to:
a wall enclosing the middle ear of the subject,
a wall enclosing the inner ear,
a walled interface between the middle and the inner ear,
a walled interface between the inner ear and the mastoid region of the subject, or
a wall of a cavity created in the mastoid region.

4. Device according to claim 2, wherein the vibrating surface is a flat surface cooperatively connected to the electromechanical actuator.

5. Device according to claim 2, wherein the vibrating surface is extended by an elongated member co-operatively connected to the electromechanical actuator.

6. Device according to claim 2, wherein the frame comprises a first sub-frame that supports the electromechanical actuator and a second sub-frame provided with the output region, wherein the vibration energy from the electromechanical actuator is directed to the output region via a vibrational-energy conducting element.

7. Device according to claim 6, wherein the conducting element is a tube adapted to contain a non-compressible liquid or gel.

8. Device according to claim 6, wherein the conducting element is a cable link, wherein the cable link comprises a flexible cable housed in a sleeve, which wherein the cable is configured to move within the sleeve while maintaining a coaxial relation therewith.

9. Device according to claim 6, wherein the conducting element is a non-flexible, elongated rod.

10. Device according to claim 6, wherein the conducting element is an adjustable telescopic slip link.

11. Device according to claim 6, wherein the conducting element is an adjustable hinged link.

12. Device according to claim 6, wherein the second sub-frame forms a passage having a receiving end to receive vibrational energy from the conducting element, and a transmitting end where vibrational energy is directed towards the inner ear fluid.

13. Device according to claim 12, wherein the second sub-frame is disposed with the vibrating surface in the passage, optionally in a region towards or at the transmitting end.

14. Device according to claim 13, wherein the vibrating surface is a flexible or flexibly suspended membrane in sealing connection with the transmitting end of the passage, and in hydraulic connection with the electromechanical actuator.

15. Device according to claim 13, wherein the vibrating surface is a flexibly suspended plate in mechanical connection with the electromechanical actuator.

16. Device according to claim 13, wherein the vibrating surface is formed from a sliding piston in hydraulic or mechanical connection with the electromechanical actuator.

17. Device according to claim 13, wherein the vibrating surface comprises:
a flexibly suspended rigid membrane in sealing connection with the transmitting end of the passage, and in hydraulic connection with the electromechanical actuator, and
a pin attached to said flexibly suspended rigid membrane.

18. Device according to claim 6, wherein the first sub-frame is configured for physical attachment to:
a wall enclosing the middle ear, or
a wall of a cavity created in the mastoid region.

19. Device according to claim 6, wherein the first sub-frame is incorporated within the a housing of a regulating unit.

20. Device according to claim 6, wherein the second sub-frame is configured for attachment at
a wall enclosing the inner ear,
a walled interface between the middle and the inner ear, or
a walled interface between the inner ear and the mastoid region.

21. Device according to claim 2, wherein the electromechanical actuator is an electromagnetic, piezoelectric, electrostatic or magnetostrictive actuator.

22. Device according to claim 6, wherein at least part of the frame, the second sub-frame, or at least part of the vibrating surface acts as the proximal electrode.

23. Device according to claim 1, wherein the proximal electrode and/or the distal electrode is pin-shaped and is configured to diverge from a longitudinal centreline of a cochlea lumen.

24. Device according to claim 1, wherein the proximal electrode, the output region, and/or the distal electrode is configured to sit flush or recessed with an inside wall of a cochlea lumen.

25. Device according to claim 1, further comprising a regulating unit configured to provide electrical signals to the proximal electrodes, the distal electrode, and/or the vibration generator, which wherein the electrical signals represent sound information.

26. Device according to claim 25, wherein the regulating unit is configured to provide full audio frequency spectrum to the vibration generator.

27. Device according to claim 25, wherein the regulating unit is configured to enhance or suppress one or more bands of audio frequency provided to the vibration generator.

28. Device according to claim 25, wherein the regulating unit is configured to translate sound information into the electrical signals for triggering nerves to fire neural signals, wherein the electrical signals are provided to the proximal electrodes and the distal electrode.

29. Device according to claim 28, wherein the regulating unit is configured to translate full audio frequency spectrum into the electrical signals.

30. Device according to claim 28, wherein the regulating unit is configured to enhance or suppress one or more bands of audio frequency and translate it into the electrical signals.

31. Device according to claim 25, wherein the regulating unit is configured to split sound information into higher frequency signals and lower frequency signals, whereby the higher frequency signals are provided to the proximal electrode and the distal electrode, and the lower frequency signals are translated and provided to the vibration generator.

32. Device according to claim 25, wherein the regulating unit is configured to receive sound information from an internal microphone, an external microphone, or a telecoil.

33. Device according to claim 25, wherein the regulating unit is configured to use measurements from a measurement electrode for closed-loop control of electrical and/or vibrational stimulation.

34. Device according to claim 25, wherein the regulating unit is configured to generate also a static pressure using the vibration generator.

35. Device according to claim 25, wherein the electromechanical actuator is configured to act as a pressure sensor.

36. Device according to claim 25, wherein the regulating unit is configured to control an inner ear pressure of the subject using the vibration generator.

37. Device according to claim 25, wherein the regulating unit comprises a receiving means for receiving sound information across a wireless link.

38. Device according to claim 25, wherein the regulating unit comprises a transmitting and/or a receiving means, configured to exchange data with an external device across a wireless link.

39. Device according to claim 25, wherein the regulating unit comprises memory storage configured to store patient-specific data.

40. Device according to claim 25, wherein the distal electrode is disposed within the regulating unit.

41. A method for improving hearing in a subject comprising the steps of:
implanting a vibration generator, comprising an output region such that the output region is located in a wall enclosing the inner ear of the subject, and applies vibrational stimulation to the inner ear fluid of the subject,
implanting a proximal electrode in a wall enclosing the inner ear, wherein the proximal electrode is proximal to the output region of vibration generator,
implanting a distal electrode such that the distal electrode makes electrical contact with the cochlea lumen of the subject.

42. Method according to claim 41, wherein the vibration generator further comprises:
an electromechanical actuator,
a vibrating surface co operatively connected to the electromechanical actuator wherein the vibrating surface provides vibrational energy, and
a frame configured to position the vibrating surface so as to direct vibrational energy therefrom to the output region.

43. Method according to claim 42, wherein the frame of the vibration generator is physically attached to:
a wall enclosing the middle ear of the subject,
a wall enclosing the inner ear,
a walled interface between the middle and the inner ear,
a walled interface between the inner ear and mastoid region of the subject, or
a wall of a cavity created in the mastoid region.

44. Method according to claim 43, wherein the frame of the vibration generator is attached so as to position the output region in a hole drilled all the way through, or drilled partially through a wall enclosing the inner ear, preferably interface between the middle and the inner ear, or preferably the interface between the inner ear and the mastoid region.

45. Method according to claim 44, wherein the hole is in a bony part.

46. Method according to claim 42, wherein the frame comprises a first sub-frame that supports the electromechanical actuator and a second sub-frame provided with the output region, wherein the vibration energy from the electromechanical actuator is directed to the output region via a vibrational-energy conducting element.

47. Method according to claim 46, wherein the first sub-frame is attached to:
a wall enclosing the middle ear of the subject, or
a wall of a cavity created in the mastoid region of the subject.

48. Method according to claim 46, wherein the first sub-frame is incorporated within a housing of a regulating unit.

49. Method according to claim 46, wherein the second sub-frame is attached to:
a wall enclosing the inner ear,
a walled interface between the middle and the inner ear, or
a walled interface between the inner ear and the mastoid region.

50. Method according to claim 43, wherein the proximal electrode is implanted at a walled interface between the middle and the inner ear.

51. Method according to claim 43, wherein the proximal electrode is implanted at a walled interface between the inner ear and the mastoid region.

52. Method according to claim 43, wherein the proximal electrode is implanted to a walled interface comprising a bony part.

53. Method according to claim 52, wherein the proximal electrode is placed in a drilled hole in the bony part, wherein the hole is drilled all the way through, or drilled partially through the bony part.

54. Method according to claim 5253, wherein the proximal electrode and the output region occupy the same said hole or occupy separately drilled holes.

55. Method according to claim 52, wherein the proximal electrode and/or the output region are placed in an oval window.

56. Method according to any of claims 41, wherein the proximal electrode and/or the distal electrode is pin-shaped and is implanted such that a longitudinal axis of the proximal electrode and/or the distal electrode diverges from a longitudinal centreline of the cochlea lumen.

57. Method according to claim 42, wherein the proximal electrode, the vibrating surface, and/or the distal electrode is implanted to be flush or recessed with an inside wall of the cochlea lumen.

58. Method according to claim 41, wherein the distal electrode is implanted such that electrical impedance between the distal electrode and the inner ear fluid at 1 kHz is from about 10 to about 10 000 ohms.

59. Method according to claim 41, wherein the distal electrode is implanted such that electrical resistance between the distal electrode and the proximal electrode is from about 10 to about 10 000 ohms.

60. Method according to claim 41, wherein the distal electrode is implanted such that electrical impedance between the distal electrode and the proximal electrode at 1 kHz is between from about 10 to about 10 000 ohms.

61. Method according to claim 41, further comprising the step of implanting a regulating unit, and connecting the proximal electrode, the distal electrode, and the vibration generator to the regulating unit using one or more connecting electrical leads.

62. (canceled)

63. A kit comprising the following components:
at least one proximal electrode,
at least one distal electrode,
at least one vibration generator,
one or more connecting electrical leads, and optionally one or more of the following:
a regulating unit,
surgical tools, and
instructions for use.

64. A kit according to claim 63, wherein the connecting electrical leads are disposed with connectors for connecting to the proximal electrode, the distal electrode, and/or the vibration generator.

65. A kit according to claim 63, wherein
the proximal electrode is configured for physical attachment to a wall enclosing the inner ear at a location proximal to the output region of the vibration generator,
the distal electrode is configured to make electrical contact with the auditory nerve, and
the vibration generator comprises an output region configured to apply vibrational stimulation to the inner ear fluid, wherein the vibration generator further comprises:
an electromechanical actuator,
a vibrating surface co-operatively connected to the electromechanical actuator, wherein the vibrating surface provides vibrational energy, and
a frame configured to position the vibrating surface to direct vibrational energy therefrom to the output region.
